(19) United States
(12) Patent Application Publication
(10) Pub. No.: US 2018/0028553 A1
(43) Pub. Date: Feb. 1, 2018

(54) USE OF STING AGONIST AS CANCER TREATMENT

(71) Applicants: The University of Chicago, Chicago, IL (US); Aduro Biotech, Inc., Berkeley, CA (US)

(72) Inventors: Thomas F. Gajewski, Chicago, IL (US); Seng-ryong Woo, Chicago, IL (US); Leticia Corrales, Chicago, IL (US); Thomas W. Dubensky, Jr., Berkeley, CA (US); David Kanne, Berkeley, CA (US); Meredith Lai Ling Leong, Berkeley, CA (US); Laura Hix Glickman, Berkeley, CA (US); Edward Emile Lemmens, Berkeley, CA (US)

(21) Appl. No.: 15/783,570

(22) Filed: Oct. 13, 2017

Related U.S. Application Data
(63) Continuation of application No. 15/035,432, filed on May 9, 2016, now abandoned.
(60) Provisional application No. 61/906,330, filed on Nov. 19, 2013.

(51) Int. Cl. A61K 31/7084; A61K 45/06; A61K 31/352

(52) U.S. Cl. CPC A61K 31/7084; A61K 31/352; A61K 45/06

(57) ABSTRACT

Methods and compositions for treating cancer by intratumorally administering a stimulator of interferon genes (STING) agonist are disclosed herein.

### CROSS-REFERENCE TO RELATED APPLICATIONS

This application is a continuation of U.S. patent application Ser. No. 15/035,432, filed May 9, 2016, which is a §371 national entry application of International Patent Application No. PCT/US2014/066436, filed Nov. 19, 2014, which claims the benefit of priority of U.S. Provisional Patent Application No. 61/906,330, filed Nov. 19, 2013, each of which is hereby incorporated by reference in its entirety.

### BACKGROUND OF THE INVENTION

### I. Field of the Invention

The present invention relates generally to the fields of biology, chemistry and medicine. More particularly, it concerns methods and compositions relating to oncology and cancer treatment.

### II. Description of Related Art

In the 1980s, it was shown that the flavone acetic acid had an antitumor effect in several tumor mouse models and produced hemorragic necrosis within the tumors. Because of its effect in the tumor vasculature, it was described as a Vascular Disrupting Agent. In addition to the effect in the vasculature, it also produced an increase in the production of several innate cytoquines.

In order to get more potent compounds, the structure of this drug was modified and the most potent derivate was DMXAA, which showed 16 times more potency in the induction of necrosis and release of cytokines. Due to the promising results in the mouse models and preclinical trials, there were some clinical trials using DMXAA for the treatment of tumors, but all failed.

In view of this, there remains the need to identify an effective manner in which to use Vascular Disrupting Agents such as DMXAA to treat cancer and tumors.

### SUMMARY OF THE INVENTION

In some embodiments, there are provided compositions and methods concerning methods for treating cancer in a subject comprising administering to the subject an effective amount of a stimulator of interferon genes (STING) agonist, wherein the STING agonist is administered intratumorally.

The STING agonist may be any appropriate agonist. In some embodiments, the STING agonist is a nucleic acid, a protein, a peptide, or a small molecule. In some embodiments, the small molecule is a cyclic dinucleotide. In some embodiments, the STING agonist is the compound:

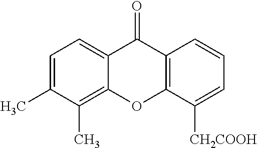

In some embodiments, the small molecule is a modified cyclic dinucleotide. In some embodiments, the modified cyclic dinucleotide may not occur in nature or may be chemically synthesized. In some embodiments, the modified cyclic dinucleotide is a compound of the formula (A):

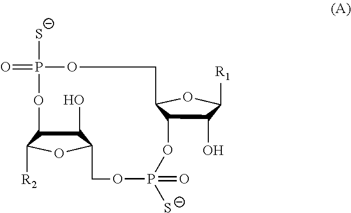

In some embodiments, R₁ and R₂ may each independently be 9-purine, 9-adenine, 9-guanine, 9-hypoxanthine, 9-xanthine, 9-uric acid, or 9-isoguanine, the structures of which are shown below, the structures of which are:

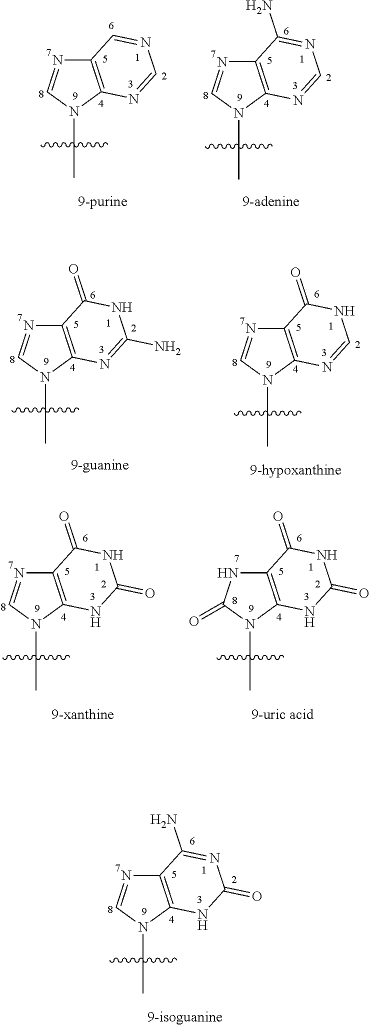

R₁ and R₂ may be identical or different. In some embodiments, the compound may be provided in the form of predominantly Rp,Rp or Rp,Sp stereoisomers, or prodrugs or pharmaceutically acceptable salts thereof. In some embodiments, the compound may be provided in the form of predominantly Rp,Rp stereoisomers. In particular embodiments, the compound may be a compound of the formula (B) below or in the form of predominantly Rp,Rp stereoisomers thereof:

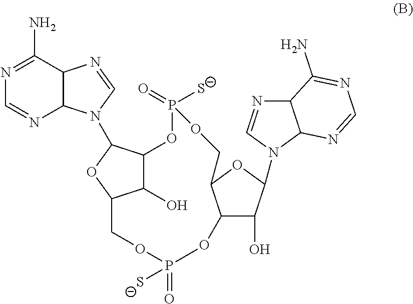 [SMILES: NC1=NC=NC2C1N=CN2C1OC2COP(=O)([S-])OC3C(O)C(COP(=O)([S-])OC2C1O)OC3N1C=NC2C(N)=NC=NC21]

In some embodiments, the compound may be dithio-(Rₚ, Rₚ)-[cyclic[A(2′,5′)pA(3′,5′)p]] (also known as 2′-5′, 3′-5′ mixed phosphodiester linkage (ML) RR-S2 c-di-AMP or ML RR-S2 CDA)) (as shown in the formula (B) above), ML RR-S2-c-di-GMP (ML-CDG), ML RR-S2 cGAMP, or any mixtures thereof.

The compounds disclosed herein have several advantages over naturally occurring cyclic dinucleotides (CDNs) or other modified CDNs because they may be able to activate one or more known human STING alleles. Further embodiments may be provided for treating cancers in a subject, comprising to the subject an effective amount of a compound as described herein. Such compounds may be used as STING agonists.

The methods of preparing such a compound may be further provided. The methods of preparing may involve at least sulfonization reactions and/or separation of RS- and RS-diastereromeres.

In further embodiments, there are provided compositions and methods concerning methods for treating cancer in a subject comprising administering to the subject an effective amount of a stimulator of interferon genes (STING) agonist, wherein the STING agonist is administered intratumorally.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease. In some embodiments, treating cancer is further defined as reducing the size of a tumor or inhibiting growth of a tumor. In particular embodiments, the subject is a human.

In some embodiments, the compositions or compounds described herein may be administered to a subject in need thereof by a variety of parenteral and nonparenteral routes in formulations containing pharmaceutically acceptable carriers, adjuvants and vehicles. Administration routes may be intratumoral or parenteral, including but, not limited to, one or more of subcutaneous, intravenous, intramuscular, intraarterial, intradermal, intrathecal and epidural administrations.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of an infection are exhibited or felt by a patient or after a clinician evaluates the patient for an infection. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of an infection (or any range derivable therein). The patient may be treated for 1, 2, 3, 4, 5, 6, 7, 8, 9, 10 or more days (or any range derivable therein) or until symptoms of an infection have disappeared or been reduced or after 6, 12, 18, or 24 hours or 1, 2, 3, 4, or 5 days after symptoms of an infection have disappeared or been reduced.

The compositions may be administered one or more times. In some embodiments, the compositions are administered 1, 2, 3, 4, 5, 6, 7, 8, 9, or 10 times or more. In specific embodiments, the STING agonist is administered 1, 2, 3, 4, 5, 6, 7, 8, 9, or 10 times or more.

Methods may be used in combination with additional cancer therapy. In some embodiments, the distinct cancer therapy comprises surgery, radiotherapy, chemotherapy, toxin therapy, immunotherapy, cryotherapy or gene therapy. In some embodiments, the cancer is a chemotherapy-resistant or radio-resistant cancer. Combination therapy may be achieved by use of a single pharmaceutical composition that includes both agents, or by administering two distinct compositions at the same time, wherein one composition includes the STING agonist and the other includes the second agent(s).

The two therapies may be given in either order and may precede or follow the other treatment by intervals ranging from minutes to weeks. In embodiments where the other agents are applied separately, one would generally ensure that a significant period of time did not expire between the time of each delivery, such that the agents would still be able to exert an advantageously combined effect on the patient. In such instances, it is contemplated that one may administer both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. In some embodiments, the STING agonist is administered prior to administration of the distinct cancer therapy. In some embodiments, the distinct cancer treatment is administered prior to administration of the STING agonist.

The cancer may be any appropriate cancer, including but not limited to melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, or hepatocellular carcinoma. In some embodiments, the cancer is melanoma. In some embodiments, the cancer is a chemotherapy or radio-resistant cancer.

“Effective amount” or “therapeutically effective amount” or “pharmaceutically effective amount” means that amount which, when administered to a subject or patient for treating a disease, is sufficient to effect such treatment for the disease. In some embodiments, the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, or 100 mg/kg (or any range derivable therein) of the agonist.

As used herein, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO_{2;} imino means ═NH (see below for definitions of groups containing the term imino, e.g., alkylamino); “cyano” means —CN; “azido” means —N₃; “mercapto” means —SH; “thio” means ═S; “sulfonamido” means —NHS(O)₂- (see below for definitions of groups containing the term sulfonamido, e.g., alkylsulfonamido); “sulfonyl” means —S(O)₂-(see below for definitions of groups containing the term sulfonyl, e.g., alkylsulfonyl); and “silyl” means —SiH₃ (see below for definitions of group(s) containing the term silyl, e.g., alkylsilyl).

For the groups below, the following parenthetical subscripts further define the groups as follows: “(Cn)” defines the exact number (n) of carbon atoms in the group; “(C≦n)” defines the maximum number (n) of carbon atoms that can be in the group; (Cn-n′) defines both the minimum (n) and maximum number (n′) of carbon atoms in the group. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3-10 carbon atoms)). Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3-10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tent-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂(methylene), —CH₂CH₂, —CH₂C(CH₃)₂CH₂-, —CH₂CH₂CH₂-, and

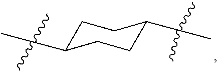 [SMILES: *C1CCC(*)CC1]

are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)-, —CF₂-, —CH(Cl)-, —CH(OH)-, —CH(OCH₃)- , and —CH₂CH(Cl).

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH-C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH-, —CH═C(CH₃)CH₂-, —CH═CHCH₂-, and

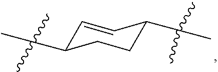 [SMILES: *C1C=CC(*)CC1]

are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH-, —C(OH)═CH-, and —CH₂CH═C(Cl)-.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and CH₂C≡CCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C-, —C≡CCH₂-, and —C≡CCH(CH₃)- are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH- and —C≡CHCH(Cl)- are non-limiting examples of substituted alkynediyl groups.

The term “aryl” when used without the “substituted” modifier refers to a monovalent group with an aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C═CH, —C₆H₄C≡CCH₃, naphthyl, and the monovalent group derived from biphenyl. The term “substituted aryl” refers to a monovalent group with an aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂N₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

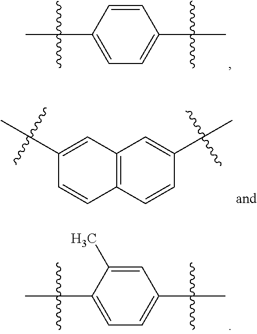

The term “substituted arenediyl” refers to a divalent group, wherein the arenediyl group is attached with two σ-bonds, with two aromatic carbon atoms as points of attachment, said carbon atoms forming part of one or more six-membered aromatic rings structure(s), wherein the ring atoms are all carbon, and wherein the divalent group further has at least one atom independently selected from the group consisting of N, O, F, Cl, Br, I, Si, P, and S.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroarenediyl” when used without the “substituted” modifier refers to a divalent group, wherein the heteroarenediyl group is attached with two σ-bonds, with an aromatic carbon atom or nitrogen atom as the point of attachment, said carbon atom or nitrogen atom two aromatic atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of heteroarenediyl groups include:

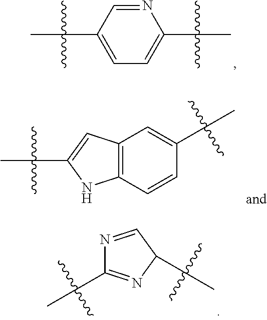

The term “substituted heteroarenediyl” refers to a divalent group, wherein the heteroarenediyl group is attached with two σ-bonds, with two aromatic carbon atoms as points of attachment, said carbon atoms forming part of one or more six-membered aromatic rings structure(s), wherein the ring atoms are all carbon, and wherein the divalent group further has at least one atom independently selected from the group consisting of N, O, F, Cl, Br, I, Si, P, and S.

The term “heteroaralkyl” when used without the “substituted” modifier refers to the monovalent group alkanediylheteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO—pyridyl, —CO—imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR′, wherein the alkylidene group is attached with one σ-bond and one π-bond, in which R and R′ are independently hydrogen, alkyl, or R and R′ are taken together to represent alkanediyl. Non-limiting examples of alkylidene groups include: ═CH₂, ═CH(CH₂CH₃), and ═C(CH₃)₂. The term “substituted alkylidene” refers to the group ═CRR′, wherein the alkylidene group is attached with one σ-bond and one π-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O—cyclopentyl, and —O—cyclohexyl. The term “substituted alkoxy” refers to the group —OR, in which R is a substituted alkyl, as that term is defined above. For example, —OCH₂CF₃ is a substituted alkoxy group.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, —N-pyrrolidinyl, and N-piperidinyl.

The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group ═NR, wherein the alkylimino group is attached with one σ-bond and one π-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one σ-bond and one π-bond, in which R is substituted alkenyl, alkynyl, aryl, aralkyl, heteroaryl, heteroaralkyl and acyl, respectively.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, —C(S)CH(CH₂)₂, 'C(S)C₆H₅, —C(S)C₆H₄CH₃, —C(S)C₆H₄CH₂CH₃, —C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, —C(S)OCH₃, —C(S)OCH₂CH₃, —C(S)OCH₂CH₂CH₃, —C(S)OC₆H₅, —C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: —S(O)₂CH₃, —S(O)₂CH₂CH₃, —S(O)₂CH₂CH₂CH₃, —S(O)₂CH(CH₃)₂, —S(O)₂CH(CH₂)₂, —S(O)₂cyclopentyl, and —S(O)₂cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylammonium” when used without the “substituted” modifier refers to a group, defined as —NH₂R⁺, —NHRR′⁺, or NRR′R″⁺, in which R, R′ and R″ are the same or different alkyl groups, or any combination of two of R, R′ and R″ can be taken together to represent an alkanediyl. Non-limiting examples of alkylammonium cation groups include: —NH₂(CH₃)⁺, —NH₂(CH₂CH₃)+, —NH₂(CH₂CH₂CH₃)+, —NH(CH₃)₂⁺, —NH(CH₂CH₃)₂⁺, —NH(CH₂CH₂CH₃)₂⁺, —N(CH₃)₃⁺, —N(CH₃)(CH₂CH₃)₂⁺, —N(CH₃)₂(CH₂CH₃)⁺, —NH₂C(CH₃)₃⁺, —NH(cyclopentyl)₂⁺, and —NH₂(cyclohexyl)⁺. The term “substituted alkylammonium” refers —NH₂R⁺, —NHRR′⁺, or NRR′R″⁺, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, W and R″ that are not either substituted alkyl or substituted alkanediyl, can be either alkyl, either the same or different, or can be taken together to represent a alkanediyl with two or more carbon atoms, at least two of which are attached to the nitrogen atom shown in the formula.

The term “alkylsulfonium” when used without the “substituted” modifier refers to the group —SRR′⁺, in which R and R′ can be the same or different alkyl groups, or R and R′ can be taken together to represent an alkanediyl. Non-limiting examples of alkylsulfonium groups include: —SH(CH₃)⁺, —SH(CH₂CH₃)⁺, —SH(CH₂CH₂CH₃)⁺, —S(CH₃)₂⁺, —S(CH₂CH₃)₂⁺, —S(CH₂CH₂CH₃)₂⁺, —SH(cyclopentyl)⁺, and —SH(cyclohexyl)⁺. The term “substituted alkylsulfonium” refers to the group —SRR′⁺, in which R and R′ can be the same or different substituted alkyl groups, one of R or R′ is an alkyl and the other is a substituted alkyl, or R and R′ can be taken together to represent a substituted alkanediyl. For example, —SH(CH₂CF₃)⁺is a substituted alkylsulfonium group.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound described herein may be replaced by a silicon atom(s). Further, it is contemplated that any oxygen atom discussed in any compound herein may be replaced by a sulfur or selenium atom.

A compound having a formula that is represented with a dashed bond is intended to include the formulae optionally having zero, one or more double bonds. Thus, for example, the structure

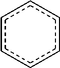

### includes the structures

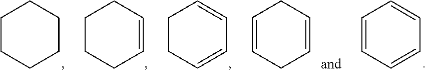

As will be understood by a person of skill in the art, no one such ring atom forms part of more than one double bond.

Any undefined valency on an atom of a structure shown in this application implicitly represents a hydrogen atom bonded to the atom.

A ring structure shown with an unconnected “R” group, indicates that any implicit hydrogen atom on that ring can be replaced with that R group. In the case of a divalent R group (e.g., oxo, imino, thio, alkylidene, etc.), any pair of implicit hydrogen atoms attached to one atom of that ring can be replaced by that R group. This concept is as exemplified below:

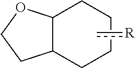

### represents

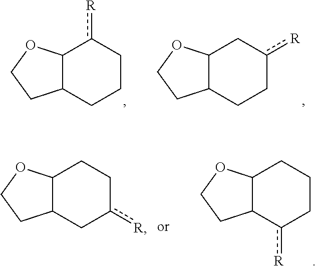

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dehydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds that are pharmaceutically acceptable, as defined above, and that possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of described embodiments is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

As used herein, “predominantly one optical isomer” means that a compound contains at least about 85% of one optical isomer (e.g., an enantiomer or diastereomer). For example, in certain embodiments, a compound may contain at least about 90% of one optical isomer. In certain embodiments, a compound may contain at least about 95% of one optical isomer. In certain embodiments, a compound may contain at least about 99% of one optical isomer. Similarly, the phrase “substantially free from other optical isomers” means that the compound contains at most about 15% of another optical isomer. For example, in certain embodiments, a compound may contain at most about 10% of another optical isomer. In certain embodiments, a compound may contain at most about 5% of another optical isomer. In certain embodiments, a compound may contain at most about 1% of another optical isomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject of patient which may be at risk and/or predisposed to the disease but does not yet experience or display any or all of the pathology or symptomatology of the disease.

“Prodrug” means a compound that is convertible in vivo metabolically into an inhibitor according to embodiments described herein. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-fl-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

The term “saturated” when referring to a atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (13 C(O)OC(CH₃)₃), benzyloxycarbonyl, p-methoxybenzyloxy carbonyl, vinyloxy carbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five, and optionally amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β, β,β-trichloroethoxycarbonyl and β-iodoethoxycarbonyl).

As used herein, the term “water soluble” means that the compound dissolves in water at least to the extent of 0.010 mole/liter or is classified as soluble according to literature precedence.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

FIG. 1—STING and IRF3 are required for CD8⁺ T cell priming in vivo. (a) CFSE-labeled 2C T cell were transferred into WT (n=6) or MyD88^{-/-} (n=6) mice and B16.SIY melanoma cells were inoculated 1 day later. After 6 days, mice were sacrificed and splenocytes were stained with anti-CD8 and the clonotypic mAb 1B2 and analyzed by flow cytometry for CFSE dilution. (b) Trif^{-/-} (n=5), (c) TLR4^{-/-} (n=4), (d) TLR9^{-/-} (n=5), (e) P2X7R^{-/-} (n=5), (f) MAVS^{-/-} (n=5), (g) STING^{-/-} (n=5) or (h) IRF3^{-/-} (n=5) mice were inoculated with 10⁶ B16.SIY melanoma cells. After 7 days, splenocytes were analyzed for SIY-specific IFN-γ-producing CD8⁺ T cell frequencies by ELISPOT assay. WT mice were used for controls. *P<0.05, ***P<0.001 (Student's t-test). Data represent mean±SEM and are representative of two (b-f) or three (a) independent experiments.

FIG. 2—Tumor-derived DNA induces IFN-β production via a STING- and IRF3-dependent pathway. (a) Cultured B16 melanoma tumor cells were treated as indicated (Methods) and incubated with BM-DCs for 18 hrs. The amount of secreted IFN-β was measured by ELISA. (b) BM-DC cells from WT or STING^{-/-} mice were stimulated with 1 μg/ml of tumor-derived DNA for indicated time points. Whole cell extracts were incubated with antibodies against pTBK1, total TBK1, pIRF3 and total IRF3. Images were acquired using Odyssey Scan (Licor) and analysed by Image Studio (Licor). (c,d) BM-DCs were generated from STING^{-/-} or IRF3^{-/-} mice and stimulated with tumor DNA using Lipofectamine. The amount of IFN-β was measured by ELISA. BM-DCs from WT mice were used as controls. (e,f) IFN-β reporter cells were transfected with siRNAs specific for STING or IRF3 followed by stimulation with tumor DNA. Reporter activity was assessed as described in Methods (e,f). **P<0.01, ***P<0.001 (Student's t-test). Data represent mean±SEM and representative of two (a) or three (b-f) independent experiments.

FIG. 3—STING^{-/-} mice are deficient at rejection of immunogenic tumors and show defective accumulation of anti-tumor T cells. (a) WT or STING^{-/-} mice (129 background) were inoculated with 10⁶ B16.SIY melanoma cells. Tumor growth was measured at indicated days. (b)After 7 days, spleens were removed and SIY-specific pentamer-specific CD8⁺ T cell frequencies were measured by flow cytometry analysis. (c) 1969 tumor cells were inoculated into WT or STING^{-/-} mice and tumor growth was recorded in indicated days. (d, e) CFSE-labeled 2C T cell were transferred into WT or STING^{-/-} mice and B16.SIY melanoma cells were inoculated into recipient mice after 1 day. On day 6, mice were sacrificed and spleens and tumor-draining lymph nodes were removed. Cells were stained with anti-CD8 and the clonotypic mAb 1B2 and analyzed by flow cytometry for CFSE dilution. *P<0.05, **P<0.01, ***P<0.001 (Students t-test). Data represent mean±SEM (n=5) and representative of two independent experiments.

FIG. 4—Tumor DNA stimulation induces a broad spectrum of genes indicative of dendritic cell activation. (a) BM-DCs from WT or STING^{-/-} mice were stimulated with tumor DNA for 7 hours and RNA was isolated. Isolated RNA was analyzed by Affymetrix GeneChip analysis described in method. (b-d) BM-DCs from WT or STING^{-/-} mice were stimulated with tumor DNA and the indicated cytokines were measured by ELISA. *P<0.05, **P<0.01 (Student's t-test). Data represent mean±SEM and are representative of three independent experiments.

FIG. 5—Tumor-infiltrating host APCs uptake tumor-derived DNA in vivo. (a,d) B16.SIY tumor cells were stained with DRAQ5 for 15 minutes. After extensive washing of tumor cells, they were inoculated into mice subcutaneously. The next day, mice were sacrificed and the tumor bump was harvested. Isolated single-cell suspensions of tumor cells were stained and single cell images were acquired using Imagestream described in the Methods. Acquired images were analyzed using IDEAS software. (b) B16.SIY tumor cells were labeled with Edu by culture of tumor cells in the presence of Edu for overnight. After washing labeled tumor cells, tumor cells were inoculated into mice. The next day, tumor bumps were harvested and Edu was detected using Click-iT Edu imaging kits (Invitrogen) described in the Methods. Acquired images were analyzed as described above. Non-labeled tumor cells were used as a negative control. (c) Human melanoma 624 tumor cells were inoculated into mice subcutaneously. The next day, tumor bumps were harvested and stained with human anti-HLA, mouse anti-CD45, and anti-CD11c antibodies. After gating live cells by DAPI staining, CD45 and CD11c positive cells were collected by cell sorting and DNA was isolated. PCR was performed using mouse (M) or human (H) specific primer for genomic (g) or mitochondrial (m) sequences as described in the Methods. (d) Sorted cells described in (c) were serially diluted (10 cells/sample) and whole genome amplification was performed using REPLI-g® Single Cell Kit (Qiagen) and PCR was performed as described in (c). ***P<0.001 (Student's t-test). Data represent mean±SEM and representative of at least three independent experiments.

FIG. 6—Tumor-infiltrating host APCs produce IFN-β in a STING-dependent fashion. (a) B16.SIY tumor cells were inoculated into mice subcutaneously. The next day, tumor bumps were harvested, and the suspended cells were fixed, permeabilized, and stained with indicated antibodies. Acquired images with imagestream were analyzed using IDEAS software. (b) B16.SIY tumor cells were inoculated into WT or STING^{-/-} mice. The next day, tumor cells, lymph nodes and spleens were isolated as above and stained with anti-mouse CD45 antibody (b) and CD11b and CD11c (c) antibodies. Stained cells were collected by cell sorting. Total RNA was isolated and cDNA was synthesized. The expression of IFN-β transcript was measured by q-PCR. CD11b- or CD11c-positive cells from lymph nodes or spleen were used for controls. *P<0.05, **P<0.01 (Student' s t-test). Data represent mean±SEM and representative of three independent experiments.

FIG. 7—Antigen-specific CD8⁺ T cell response in TLR4^{-/-} and TLR9^{-/-} mice is comparable to WT Mice. B16.SIY melanoma cells were injected into WT or TLR4^{-/-} (a) or TLR9^{-/-} (b) mice. After 1 week, the spleen of mice was isolated and SIY peptide-specific pentamer staining was performed as described in Methods. Data represent mean±SEM and representative of two independent experiments.

FIG. 8—Tumor-derived DNA induces production of IFN-β in mouse macrophage cells. Immortalized macrophage cell lines were stimulated with either tumor-derived DNA+Lipofectamine, live tumor cell, or culture supernatant of B16 tumor cells and the amount of produced IFN-β was measured by ELISA.

FIG. 9—DNA from normal splenocytes induced production of IFN-β comparable to tumor derived DNA. DNA from spleen of WT B6 mice or B16 melanoma tumor cells was isolated using the DNA isolation kit (Qiagen). BMDCs were stimulated with indicated concentrations of DNA and IFN-β production was measured from cell culture supernatants by ELISA. Data represent mean±SEM and representative of three independent experiments.

FIG. 10—Tumor-derived DNA stimulation induces phosphorylation of TBK1 and IRF3 in WT BMDCs not in STING^{-/-} BMDCs. BMDCs were stimulated with either tumor-derived DNA (1 μg/ml) or LPS (20 ng/ml) for indicated times. Whole cell extracts were incubated with antibodies against pTBK1, total TBK1, pIRF3 and total IRF3. Images were acquired using Odyssey Scan (Licor) and analysed by Image Studio (Licor).

FIG. 11—DNA stimulation appears not to induce substantial NF-Kb activation. BM-DC cells from WT mice were stimulated with 1μg/ml of tumor-derived DNA, 20 ng/ml LPS for different time points. Whole cell extracts were analyzed with antibodies against pIKKβ, total IKKβ, pIkBα and total IkBα. Data are representative of three independent experiments.

FIG. 12—cGAS knock down decreases IFN-β production from murine macrophage cells stimulated with DNA. Murine macrophage cells were treated with control or cGAS-specific siRNAs. After 36hrs, siRNA-treated cells without tumor DNA stimulation were used for RNA isolation and gene expression check by qRT-PCR (a). Another set of siRNA treated cells were stimulated with tumor DNA and production of IFN-β was measured by ELISA in cell culture supernatants (b). Data represent mean±SEM and are representative of three independent experiments.

FIG. 13—B16 melanoma tumor growth was more accelerated in STING^{-/-} (a) or IRF3^{-/-} mice (b) but not in Trif^{-/-} mice (c) compared to WT mice. B16.SIY tumor cells (10⁶ cells/mouse) were injected into the indicated mice subcutaneously and tumor growth was measured at the indicated days. Data represent mean±SEM and representative of two independent experiments.

FIG. 14—STING^{-/-} mice reject skin grafts with similar kinetics as wild type recipients. Skin from male STING^{-/-} mice was transplanted into female STING^{-/-} recipients (n=6). Wildtype male into female skin was used as a positive control (n=3). Percent surviving grafts was assessed over time.

FIG. 15—DNA of 1969 tumor cells can be transferred to host APCs in vivo. 1969 tumor cells were labeled with Edu and injected into mice. After 1 day, tumor cells including tumor-infiltrating immune cells were isolated and stained for cell surface marker and Edu as described in Methods. Images were taken by Amnis ImageStream system and data were analyzed using IDEAS software. Data shows one representative set of images of two independent experiments.

FIG. 16—No detection of human genomic DNA sequences in sorted mouse CD45⁺CD11c⁺cells. Human melanoma 624 cells were injected into mice subcutaneously. The next day, the tumor bump was isolated and single cell suspensions were prepared. After staining with DAPI, anti-human HLA (Alexa fluor 488), anti-mouse CD45 (PE), and anti-mouse CD11 c (Percp-Cy5.5) antibodies, live anti-human HLA⁻ anti-mouse CD45⁺CD11c⁺ cells were purified by cell sorting. DNA was isolated from sorted cells and PCR was performed with the indicated primer sets which are specific for human genomic DNA (STING, AIM-2 and ATG14) and mitochondrial DNA (ATP6). PCR products were electrophoresed in 1.5% agarose gel and visualized with EtBr.

FIG. 17—Mitochondrial DNA induces production of Type I IFN. Genomic DNA was isolated from B16 melanoma cells using Blood & Cell Culture DNA Midi Kit (Qiagen). Mitochondrial DNA was isolated from mitochondria of B16 melanoma cells using Qproteome™ Mitochondria Isolation kit (Qiagen). (a). THP-1 ISG reporter cells (Invivogen) were stimulated with the indicated amount of genomic or mitochondrial DNA combined with Lipofectamine (0.5 μl/well). After overnight incubation, supernatant was collected and QUANTI-blue substrate (Invivogen) was added. The amount of type I interferon production was measured by reading absorbance with a plate reader. (b). BMDCs were stimulated with indicated amount of DNA and IFN-β was measured by mouse IFN-β ELISA. Data represent mean±SEM and are representative of three to four independent experiments.

FIG. 18—Tumor-infiltrating host APCs show phosphorylation of TBK1 in vivo. B16 melanoma cells were injected into mice. After 1 day, tumor cells including tumor-infiltrating host immune cells were isolated and stained as described in Methods. Images were acquired using the Amnis ImageStream system and data were analyzed using IDEAS software. Data show images of one representative of two independent experiments.

FIG. 19—Tumor-infiltrating host APCs show phosphorylation of IRF3 at 1 week after tumor injection in vivo. B16 melanoma cells were injected into mice. After 1 week, tumor cells including tumor-infiltrating host immune cells were isolated and stained as described in Methods. Images were acquired using the Amnis ImageStream system and data were analyzed using IDEAS software. Data show images from one representative of two independent experiments.

FIG. 20 DMXAA activates the STING pathway and triggers type I IFN production.

FIG. 21 Induction of cytokines in BM-DC by DMXAA is STING-dependent.

FIG. 22 Induction of costimulatory ligands in BM-DC by DMXAA is STING-dependent.

FIG. 23 Intratumoral DMXAA triggers rejection of B16.SIY tumors in WT mice.

FIG. 24 DMXAA triggers a potent CD8⁺ T cell response against the tumor-expressed SIY antigen.

FIG. 25 DMXAA protects animals against a second tumor rechallenge.

FIG. 26 DMXAA fails to control tumor growth in STING^{-/-} and RAG^{-/-} mice.

FIG. 27 DMXAA triggers rejection of B16.SIY tumors in WT mice.

FIG. 28 DMXAA triggers a potent immune response against SIY antigen.

FIG. 29. DMXAA activates the STING pathway and promotes the activation of APCs. (a) STING^{-/-} mouse bone marrow-derived macrophages (BMM) transduced to express STING-HA tag were stimulated for 1 hour with 50 μg/ml DMXAA, stained with specific antibodies against HA tag, CD11b and DAPI. Single cell images were acquired in the ImageStream and data were analyzed with the IDEAS software (Amnis, Millipore). The data in the graph represent average of percentage of cells with aggregates from three independent experiments. (b) WT or STING^{-/-} BMM were stimulated with 50 μg/ml of DMXAA for the indicated time points. The amount of pTBK1, total TBK1, pIRF3, total IRF3, STING and GAPDH was measured by Western blot. (c) WT or STING^{-/-} BMM were stimulated with 50 μg/ml of DMXAA for 12 hours. The amount of secreted IFN-β was measured by ELISA. (d) Bone marrow-derived DCs (BM-DC) from WT or STING^{-/-} mice were stimulated with 25 μg/ml of DMXAA for the indicated time points. The amount of pTBK1, total TBK1, pIRF3, total IRF3 and GAPDH was measured by Western blot. (e) BM-DCs from WT or STING^{-/-} mice were stimulated with 50 μg/ml of DMXAA for 12 hours. The amount of IFN-β in the supernatant was measured by ELISA. (f-g) BM-DCs from WT or STING^{-/-} mice were stimulated with 25 μg/ml of DMXAA for 4h. Expression of innate cytokines was measured by q-RT-PCR (f); expression of co-stimulatory molecules on the cell membrane was measured by staining with specific antibodies against CD11c, CD40, CD86 and MHC class II (g). Cells were acquired in the LSRII-Blue Cytometer and analyzed with the FlowJo software (Treestar).

FIG. 30. Rejection of tumors in response to DMXAA is STING-dependent. (a) WT C57BL/6 mice were inoculated with 10⁶ B16.SIY cells in the left flank (n=5). When tumor volumes were 100-200 mm³ they received a single IT dose of 500 μg of DMXAA or saline. Tumor volume was measured at the indicated time points. (b-c) WT C57BL/6 mice were treated as in (a) and 5 days later splenocytes were harvested and re-stimulated in vitro in the presence of culture medium or soluble SIY peptide for 16 hours. The frequency of tumor-specific IFN-γ-producing cells was assessed by ELISPOT (b), and the percentage of SIY-specific CD8⁺ T cells was assessed by staining splenocytes with antibodies against TCRβ, CD4, CD8 and SIY pentamer (c). Cells were acquired in the LSRII-Blue cytometer and analyzed with FlowJo software. Results are shown as mean±s.e.m. **P<0.01; ***P<0.001 (c). (d) WT mice that had rejected B16.SIY tumors were rechallenged with 10⁶ B16.SIY in the contralateral flank. Nave mice were used as controls. Tumor size was measured at the indicated time points. (e) WT mice were inoculated with 10⁶ B16.SIY cells in the left and the right flanks (n=5). When tumor volumes were 100-200 mm³, 500 ₗμg of DMXAA or saline was injected IT only in the right flank and tumor volume was measured at the indicated time points. Data are representative of at least three independent experiments, or two independent experiments for the contralateral tumor model. Results are shown as mean tumor volume±s.e.m. *P<0.5; **P<0.01; ***P<0.001.

FIG. 31. The adaptive immune response is required for the majority of the therapeutic effect of DMXAA in vivo. (a) WT and STING^{-/-} C57BL/6 mice were inoculated with 10⁶ B16.SIY cells in the left flank (n=5). When tumor volumes were 100-200 mm³ they received a single IT dose of 500 μg of DMXAA or saline. Tumor size was measured at different time points. (b-c) WT and STING^{-/-} C57BL/6 mice were treated as in (a) and 5 days later splenocytes were harvested and re-stimulated in vitro in the presence of culture medium or soluble SIY peptide for 16 hours. The frequency of tumor-specific IFN-γ-producing cells was assessed by ELISPOT (b), and the percentage of specific SIY CD8⁺ T cells was assessed by staining splenocytes with specific antibodies against TCRβ, CD4, CD8 and SIY pentamer (c). Cells were acquired in the LSRII-Blue cytometer and analyzed with the FlowJo software. Results are shown as mean±s.e.m. *P<0.5; **P<0.01. WT and RAG^{-/-} C57BL/6 mice (d) or WT and TCRα^{-/-} mice (e) were inoculated with 10⁶ B16.SIY cells in the left flank (n=5). When tumor volumes were 100-200 mm³ they received a single IT dose of 500 μg of DMXAA or saline. Tumor volume was measured at the indicated time points. (f) WT C57BL/6 mice were depleted of CD8⁺ T cells by a weekly injection of 250 μg of anti-CD8 antibody (clone 2.43); isotype IgG2b was used as control. Two days after the first injection of anti-CD8 or IgG2b isotype control mice were challenged with 10⁶ B16.SIY cells in the left flank (n=5). Seven days later, when tumors were 100-200 mm³ they received a single IT dose of 500 μg of DMXAA or saline. Tumor volume was measured at different time points. Data are representative of at least two independent experiments. Results are shown as mean tumor volume±s.e.m. *P<0.5; **P<0.01; ***P<0.001.

FIG. 32. Modified CDNs potently activate STING and signal through all human STING alleles. (a) Domain structure of hSTING is shown with the positions of the amino acid variations (bottom). The allelic frequencies of the hSTING isoforms shown on the left hand column were obtained from the 1000 Genome Project database as previously described ³⁵. Whole cell lysates from HEK 293T cells stably expressing the indicated full length STING-HA proteins were analyzed by Western blot with anti-HA antibodies. (b) HEK 293T cells stably expressing the indicated STING alleles were transfected with an IFN-β-luciferase reporter construct. After 24 hours, cells were stimulated for 6 hours with 100 μg/ml DMXAA before measuring luciferase gene reporter activity. (c) HEK 293T cells expressing the indicated STING alleles were treated as in (b), stimulated for 6 hours with the indicated CDN compound (10 μM), and assessed for IFN-β-reporter activity. (d) CDNs were added to BMMs isolated from C57BL/6 or from STING^{-/-} mice at 5 μM. After a 6 hour incubation, induced expression of IFN-β was assessed by real-time qRT-PCR, and relative normalized expression was determined by comparison with unstimulated C57BL/6 BMMs. (e) Human PBMCs from donors with the indicated STING alleles were stimulated with 10 μM of the indicated CDN, or 100 μg/ml DMXAA. After a 6-hour stimulation, fold-induction of IFN-β was measured by qRT-PCR and relative normalized expression was determined by comparison with untreated controls.

FIG. 33. Synthetic CDN modifications significantly improve anti-tumor efficacy in established B16 tumors. WT C57BL/6 mice were inoculated with 5×10⁴ B16.F10 cells in the left flank (n=8). When tumor volumes were 100 mm³ they received three 25 μg IT doses of ML-CDA, ML-CDG, ML RR-S2 CDG, or ML RR-S2 CDA (a), three IT doses of DMXAA (150 μg), ML RR-S2 CDG (25 μg) or ML RR-S2 CDA (50 μg) (b), or three IT doses of ML-cGAMP (50 μg), ML RR-S2 cGAMP (50 μg), ML RR-S2 CDG (25 μg) or ML RR-S2 CDA (50 μg) (c). Control groups were treated with HBSS vehicle. (d) WT C57BL/6 mice or STING^{-/-} mice were treated with three IT doses of CDN ML RR-S2 CDA (50 μg), murine type B CpG ODN 1668 (50 μg), or HBSS vehicle control. (e) WT C57BL/6 mice were treated with three IT doses of ML RR-S2 CDA (50 μg), or 50 μg of the following TLR agonists: TLR 3 (and RIG-I) agonist, poly I:C; TLR 4 agonist, glucopuranosyl lipid A (GLA); TLR 7/8 agonist, resiquimod (R848); TLR 9 agonist CpG 1668. Compounds were administered on the days indicated by the arrows and tumor measurements were taken twice weekly. Data are representative of at least two independent experiments. Results are shown as mean tumor volume±s.e.m. *P<0.05, **P<0.01, ***P<0.001.

FIG. 34 ML RR-S2 CDA promotes immune-mediated tumor rejection. (a) WT BALB/c mice were inoculated with 10⁵ CT26 colon carcinoma cells in the left flank. When tumor volumes were 100 mm³ they received three doses IT of ML RR-S2 CDA (50 μg), or HBSS vehicle control (left graph). Mice were re-implanted with 10⁵ tumor cells on the opposite flank on day 55 post-initial tumor implantation. Naïve mice were used as controls (right graph) (n=8). (b) WT BALB/c mice were inoculated with 10⁵ CT26 colon carcinoma cells in the left flank and treated on days 11, 14, and 18 with IT injections of ML RR-S2 CDG or ML RR-S2 CDA (25 μg each), or HBSS vehicle control (n=4). 21 days post-implantation of CT26 tumors, PBMCs were stimulated with AH1 (gp70₄₂₃₋₄₃₁) and assessed by IFN-γ ELISPOT assay. (c) WT BALB/c mice were implanted with 10⁵ of CT26 tumor cells on both flanks. On the days indicated, mice were treated in one flank only with ML RR-S2 CDA (50 μg), or HBSS vehicle control (n=8). (d) WT C57BL/6 were inoculated with 5×10⁴B16.F10 melanoma cells on the right flank at day 0, and implanted IV with 10⁵ cells on day 7. Naïve mice were implanted with cells IV only as a control. Flank tumors were treated on the days indicated with ML RR-S2 CDA (50 μg), DMXAA (150 μg) or HBSS control (n=8). On day 28, lungs were harvested and enumerated for lung tumor nodules. The histogram depicts total numbers of lung tumor nodules in the ML RR-S2 CDA, DMXAA or HBSS control treated mice, compared to the untreated IV only tumor implanted mice. The images depict the ML RR-S2 CDA and HBSS control treated mice. Data are representative of at least two independent experiments. Results are shown as mean±s.e.m. **P<0.01, ***P<0.001.

FIG. 35 DMXAA dose-response in vivo. (a) WT C57BL/6 mice were inoculated with 106 B16.SIY cells in the left flank (n=5). When tumor volumes were 100-200 mm³ they received a single dose IT of 625, 500, 300 or 150 μg of DMXAA, or saline. Tumor volume was measured at different time points. Results are shown as mean tumor volume±s.e.m. (b) WT C57BL/6 mice were treated as in (a) and 5 days after DMXAA treatment, splenocytes were harvested and restimulated in vitro in the presence of culture medium or soluble SIY peptide for 16 hours. The frequency of tumor-specific IFN-γ producing cells was assessed by ELISPOT. (c) The percentage of SIY specific CD8+ T cells was assessed by staining splenocytes with specific antibodies against TCRβ, CD4, CD8 and SIY tetramer. Cells were acquired in the LSRII-Blue cytometer and analyzed with the FlowJo software. Data represent at least two independent experiments.

FIG. 36 Therapeutic effect of DMXAA in different mouse tumor models. WT mice were inoculated with 106 B16.F10 (a) TRAMP-C2 (b) into C57BL/6 mice; 4T-1 into BALB/C mice (c) and Ag104L into C3H mice (d). When tumor volumes were 100-200 mm3 they received a single IT dose of 500 μg of DMXAA or saline. Tumor volume was measured at different time points. Results are shown as mean tumor volume±s.e.m. Data represent at least two independent experiments.

FIG. 37 Frequency of CD8+ T cell in the blood of mice treated with anti-CD8. WT C57BL/6 mice were depleted of CD8 by a weekly injection of 250 μg of anti-CD8 antibody (clone 2.43) as indicated by the arrows, isotype IgG2b was used as control. The graph represents the percentage of CD8+ cells gated from TCRβ+ cells in the blood at days 0, 2, 9 and 13.

FIG. 38 Structure of cyclic dinucloetides. (a) (Upper panel) HPLC chromatograph of ML-RR-CDA purification to ≧95%, using a 2% to 20% acetonitrile gradient in 10 mM triethylammonium acetate C-18 column, showing retention time of 12.40 min. (Lower panel) Two-dimensional 1H-31P Heteronuclear Multiple Bond Correlation (HMBC) of synthesized ML RR-S2 CDA. Two dimensional 1H-31P HMBC revealed that the phosphorus nucleus, P-1 is correlated to the 2′-ribose proton (H-2A) as well as the 5′ ribose methyelene protons (H-5B). The other phosphorous nucleus, P-2, is correlated to the 3′ ribose proton (H-3B) and to the 5′ ribose methyelene protons (H-5A) of the other adenosine. The combined 1H-1H COSY and 2D-HMBC results provide direct evidence that the regiochemistry of the phosphodiester linkages is 2′,5′-3′,5′according to the structure shown. (b) (Upper panel) X-ray crystallographic structure (stick model) of ML RR-S2 CDA, confirming the R,R diastereomer configuration and regiochemistry of the 2′-5′-3′-5′ phosphodiester linkages. Color scheme: carbon (white); nitrogen (blue); oxygen (red); sulfur (yellow). (Lower panel) Electrostatic surface potential of ML RR-S2 CDA displayed with green (positive), yellow (neutral), and red (negative).

FIG. 39 Induction of pro-inflammatory cytokines by CDNs is STINGdependent. CDNs were added to BMMs isolated from C57BL/6 or from STING−/+ mice at 5 μM. After 6 hour incubation, induced expression of CCL2/MCP-1, TNF-α and IL-6 proinflammatory cytokines was assessed by real-time quantitative RT-PCR, and relative normalized expression was determined by comparison with unstimulated C57BL/6 BMMs, and GUSB and PGK1 reference genes.

FIG. 40 Activation of the STING pathway by cyclic dinucleotides. (a) STING−/− macrophages expressing STING-HA were stimulated for 1 hour with 50 mg/ml DMXAA or 50 μM ML RRS2 CDA then stained with specific antibodies against HA tag, CD11b and DAPI. Single cell images were acquired in the ImageStream and data were analyzed with the IDEAS software (Amnis, Millipore). (b) WT macrophages were stimulated with 50 μg/ml of DMXAA or 50 μM ML RR-S2 CDA for the indicated time points. The amount of pTBK1, total TBK1, pIRF3, total IRF3, STING and GAPDH was measured by Western blot. (c) BM-DCs derived from WT or STING−/− mice were stimulated in media with 10 μM of the indicated CDNs, 1 μg/ml LPS, or 100 μg/ml DMXAA. After 24 hours, expression of MHC class II or CD86 was measured by FACS gated on CD11c+ DCs.

FIG. 41 Lead CDN molecule promotes immune-mediated tumor rejection in the 4T-1 mouse model. WT BALB/c mice were inoculated with 105 4T-1 cells in the left flank. When tumor volumes were 100 mm3 they received three doses IT of ML RR-S2 CDA (50 μg) or HBSS vehicle control. Mice were re-implanted with tumor cells (1×10⁵ each) on the opposite flank on day 55 post-initial tumor implantation. Naïve control mice were also implanted at the same time (right graph) (n=8). (b) WT BALB/c mice were implanted with 1×10⁵ of 4T-1 tumor cells on both flanks. On the days indicated, mice were treated in one flank only with ML RR-S2 CDA (50 μg), or HBSS vehicle control (n=8). Data are representative of at least two independent experiments. Results are shown as mean±s.e.m. **P<0.01.

### DETAILED DESCRIPTION OF THE INVENTION

The inventors determined that the xanthenone derivative DMXAA, a stimulator of interferon genes (STING) agonist, triggers a potent activation of the STING pathway in APCs that led to high production of IFN-β, and also IL-6, TNF-α, and IL-12; and upregulation of CD40 and CD86 by DCs. Similarly, a single intratumoral dose of DMXAA promotes the rejection of established tumors in mice.

It was also discovered that complete tumor rejection depends on host STING and an adaptive T cell response, and treatment of tumors with DMXAA potently enhances the T cell immune response and generates immunologic memory. Tests were performed in both mice models and using the human molecule.

A. STING Pathway

The STING pathway is a pathway that is involved in the detection of cytosolic DNA. Stimulator of interferon genes (STING), also known as transmembrane protein 173 (TMEM173) and MPYS/MITA/ERIS, is a protein that in humans is encoded by the TMEM173 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

STING is encoded by the TMEM173 gene. It works as both a direct cytosolic DNA sensor (CDS) and an adaptor protein in Type I interferon signaling through different molecular mechanisms. It has been shown to activate downstream transcription factors STAT6 and IRF3 through TBK1, which are responsible for antiviral response and innate immune response against intracellular pathogen.

STING resides in the endoplasmic reticulum, but in the presence of cytosolic DNA, the sensor cGAS binds to the DNA and forms cyclic dinucleotides. This di-nucleotide binds to STING and promotes its aggregation and translocation from the ER through the Golgi to perinuclear sites. There, STING complexes with TBK1 and promotes its phosphorylation. Once TBK1 is phosphorylated, it phosphorylates the transcription factor IRF3, which dimerizes and translocates to the nucleus, where it activates the transcription of type I IFN and other innate immune genes.

B. STING Agonists

In some embodiments, disclosed herein are agonists that directly activates this pathway, including but not limited to DMXAA or cyclic dinucleotides or any derivatives thereof, discussed in detail below.

### 1. DMXAA

It has previously been shown that the flavone acetic acid had an antitumor effect in several tumor mouse models and produced hemorragic necrosis within the tumors. Because of its effect in the tumor vasculature, it was described as a Vascular Disrupting Agent. But apart from the effect in the vasculature, it also produced an increase in the production of several innate cytoquines.

Vadimezan or ASA404 (also known as DMXAA) is a tumor-vascular disrupting agent (tumor-VDA) that attacks the blood supply of a cancerous tumor to cause tumor regression. This flavone acetic acid derivative [5,6-dimethylXAA (xanthenone-4-acetic acid)] displays vascular-disrupting activity and induced haemorrhagic necrosis and tumour regression in pre-clinical animal models. Both immune-mediated and non-immune-mediated effects contributed to the tumour regression.

DMXAA has the following structure:

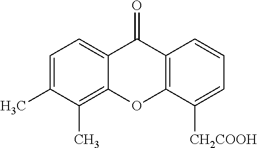

### 2. Cyclic Dinucleotides or Derivaties Thereof

The STING signaling pathway is activated by cyclic dinucleotides (CDNs), which may be produced by bacteria or produced by antigen presenting cells in response to sensing cytosolic DNA. Unmodified CDNs have been shown to induce type I interferon and other co-regulated genes, which in turn facilitate the development of a specific immune response.

In particular embodiments, the cyclic dinucleotides may include modified cyclic dinucleotides, such as a compound of the formula:

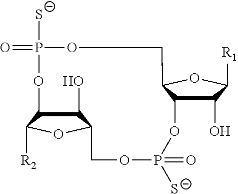

The compound may not occur in nature or may be chemically synthesized. In further embodiments, R1 and R2 may be independently 9-purine, 9-adenine, 9-guanine, 9-hypoxanthine, 9-xanthine, 9-uric acid, or 9-isoguanine, as shown below.

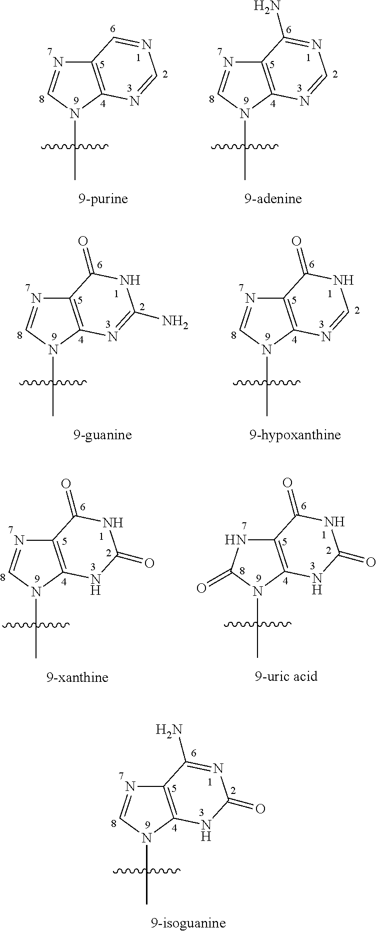

In some embodiments, the compound may be provided in the form of predominantly Rp,Rp or Rp,Sp stereoisomers, or prodrugs or pharmaceutically acceptable salts thereof. In some embodiments, the compound may be provided in the form of predominantly Rp,Rp stereoisomers. In some embodimemts, the compound may be a compound of the formula or in the form of predominantly Rp,Rp stereoisomers thereof:

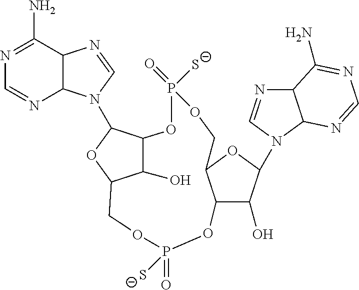

In some embodiments, the compound may be dithio-(Rₚ, Rₚ)-[cyclic[A(2′,5′)pA(3′,5′)p]] (also known as 2′-5′, 3′-5′ mixed phosphodiester linkage (ML) RR-S2 c-di-AMP or ML RR-S2 CDA)), ML RR-S2-c-di-GMP (ML-CDG), ML RR-S2 cGAMP, or any mixtures thereof.

C. Pharmaceutical Compositions and Methods

In some embodiments, pharmaceutical compositions are administered to a subject. Different aspects involve administering an effective amount of a composition to a subject. In some embodiments, a composition comprising an inhibitor may be administered to the subject or patient to treat cancer or reduce the size of a tumor. Additionally, such compounds can be administered in combination with an additional cancer therapy.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein. In specific embodiments, 50 mg/10 mL (5 mg/mL) of the inhibitor ipilimumab is administered. In specific embodiments, 200 mg/40 mL (5 mg/mL) of the inhibitor ipilimumab is administered.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of an infection are exhibited or felt by a patient or after a clinician evaluates the patient for an infection. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of an infection (or any range derivable therein). The patient may be treated for 1, 2, 3, 4, 5, 6, 7, 8, 9, 10 or more days (or any range derivable therein) or until symptoms of an infection have disappeared or been reduced or after 6, 12, 18, or 24 hours or 1, 2, 3, 4, or 5 days after symptoms of an infection have disappeared or been reduced. In specific embodiments, the inhibitor ipilimumab is administered every three weeks.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplastic disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma. The cancerous conditions amenable for treatment include metastatic cancers. “Treatment” as used herein refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, reduction of any direct or indirect pathological consequences of the disease, decreasing the rate of disease progression, amelioration or palliation of the disease state, and remission or improved prognosis. In some embodiments, the compositions are used to delay development of a disease or disorder. In non-limiting examples, the compositions may be used to reduce the rate of tumor growth or reduce the risk of metastasis of a cancer.

“Treatment” as used herein refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, reduction of any direct or indirect pathological consequences of the disease, decreasing the rate of disease progression, amelioration or palliation of the disease state, and remission or improved prognosis. In some embodiments, the compositions of the invention are used to delay development of a disease or disorder. In non-limiting examples, the compositions may be used to reduce the rate of tumor growth or reduce the risk of metastasis of a cancer.

The compositions disclosed herein can be used either alone or in combination with other compositions in a therapy. For instance, a composition may be co-administered with chemotherapeutic agent(s) (including cocktails of chemotherapeutic agents), other cytotoxic agent(s), anti-angiogenic agent(s), cytokines, thrombotic agents, and/or growth inhibitory agent(s). Such combined therapies noted above include combined administration (where the two or more agents are included in the same or separate formulations), and separate administration, in which case, administration of the antibody can occur prior to, and/or following, administration of the adjunct therapy or therapies.

Combination therapy may be achieved by use of a single pharmaceutical composition that includes both agents, or by administering two distinct compositions at the same time, wherein one composition includes the antibody and the other includes the second agent(s).

The two therapies may be given in either order and may precede or follow the other treatment by intervals ranging from minutes to weeks. In embodiments where the other agents are applied separately, one would generally ensure that a significant period of time did not expire between the time of each delivery, such that the agents would still be able to exert an advantageously combined effect on the patient. In such instances, it is contemplated that one may administer both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

The compositions may also be administered in combination with radiotherapy, surgical therapy, immunotherapy (particularly radioimmunotherapy), gene therapy, or any other therapy known to those of ordinary skill in the art for treatment of a disease or disorder associated with vascular proliferation, such as any of the diseases or disorders discussed elsewhere in this specification.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the claims.

### Example 1

### Materials and Methods

Mice and cells. C57BL/6, 129, MyD88^{-/-}, Trif^{-/-}, P2X7R^{-/-}, IPS-1^{-/-}, TLR4^{-/-}, TLR9^{-/-}, Tmem173^{-/-}(STING-deficient), Irf3^{-/-}, and 2C TCR Tg mice were used. The C57BL6-derived melanoma cell line B16.F10.SIY (henceforth referred to as B16.SIY) was used (Fuertes, et al. 2011). All cells were cultured in complete DMEM media supplemented 10% heat-inactivated FCS. For measurement of type I interferon reporter activity, B16-Blue™ IFN-α/β reporter cells were purchased from InvivoGen and maintained according to the manufacturer's instructions.

2C CD8⁺ T cell purification, CFSE staining and adoptive transfer. 2C TCR Tg CD8⁺ T cells were isolated from spleens and lymph nodes of 2C/RAG2^{-/-} mice by using magnetic beads. T cells were loaded with 2.5 mM CFSE and transferred into WT or designated gene-targeted mice (4×10⁶ cells/mouse). After 1 day, recipient mice received 10⁶ B16.SIY cells, and 5 days later splenocytes from recipient mice were analyzed after staining with anti-mouse CD8α-APC and biotin-labeled anti-2C-TCR (1B2) with SA-PE by flow cytometry to assess CFSE dilution.

IFN-γ ELISPOT and pentamer staining. Splenocytes were plated at 10⁶ cells/well and stimulated overnight with SIY peptide (80 nM) or PMA (50 ng/ml) plus ionomycin (0.5 μM) as a positive control. Spots were developed using the BD mouse IFN-γ kit and the number of spots was measured using an Immunospot Series 3 Analyzer and analyzed using ImmunoSpot software (Cellular Technology Ltd). For pentamer staining, cells were labeled with PE-MHC class I pentamer (Proimmune) consisting of murine H-2K^{b} complexed to SIYRYYGL (SIY) peptide anti-CD8-APC (53-6.7), anti-CD19-PerCP-Cy5.5 (6D5), and anti-CD4-PerCP-Cy5.5 (RM4-5). Stained cells were analyzed using FACSCanto or LSR II cytometers with FACSDiva software (BD). Data analysis was conducted with FlowJo software (Tree Star).

Preparation of B16 melanoma extracts for potential IFN-β induction in vitro. For generation of B16 melanoma-derived extracts, cultured tumor cells were treated with staurosporin (0.5 μM) for 4 hrs, or irradiated (15,000 rad), or incubated for 1 hr at 55° C. for heat killing, or mechanically killed using 10 passages through a syringe and needle, or treated 3 times by freezing/thawing cycles using liquid nitrogen and water bath at 37° C. For tumor-derived genomic DNA isolation, B16 tumor cells were washed with DMEM and DNA was isolated using Blood & Cell Culture DNA Midi Kit (Qiagen). For mitochondrial DNA isolation, mitochondrias from B10 melanoma cells were isolated with Qproteome™ Mitochondria Isolation kit (Qiagen) and then mitochondrial DNA was isolated using QlAprep® Spin Miniprep Kit (Qiagen). The concentration/purity of DNA was determined by NanoDrop 1000 (Thermo Scientific). Each cell extract was added into BM-DCs and incubated for 18 hrs at 37° C. and amount of IFN-β was measured by ELISA.

In vitro IFN-β measurement. IFN-β reporter cell line was cultured in 96-well plates and stimulated with tumor-derived DNA (20 ng/well) with Lipofectamine™2000 (0.75 μl/well) (Invitrogen) for 18 hrs. Bone marrow-derived dendritic cells (BMDCs) were generated by culturing bone marrow cells in the presence of rmGM-CSF (20 ng/ml; BioLegend) for 9 days followed by stimulation with tumor-derived DNA (20 ng/well) for 7 hours. After incubation, supernatant was collected and IFN-β was measured by ELISA (PBL interferon source) or adding substrate (QUANTI-Blue;InvivoGen) for the reporter cell line.

Western blot and siRNA-mediated interference. WT or STINGko BM-DCs were stimulated with 1 μg/ml of tumor-derived DNA for 1, 3 or 6 hours. Proteins were extracted with Triton-X buffer (150 mM sodium chloride, 50 mM Tris, 1% Triton-X, pH 8.0) with proteinase inhibitors (Thermo scientific) and phosphatase inhibitors (Sigma). 30 μg of protein was electrophoresed in 10% SDS-PAGE gels and transferred onto Immobilon-FL membranes (Millipore). The blots were incubated with antibodies specific for phosphorylated TBK1 (Ser172), phosphorylated IRF3 (Ser396), total TBK1, and total IRF3 (all antibodies from CellSignaling, except anti-total IRF3 from Invitrogen). Anti-rabbit IRDye 680RD label secondary antibody was used for visualization of bands in the Odyssey

Scan (Licor) and densitometry of each band was calculated using Li-cor software.

The siRNAs for STING and IRF3 were purchased from Invitrogen (Silencer® Select siRNA). IFN-β reporter cells were cultured in 96-well plates at a density of 5×10⁴ cells per well and transfected with siRNA targeting mouse IRF-3 (sense strand: 5′-GGAAAGAAGUGUUGCGGUUtt-3′ [SEQ ID NO. 1]), mouse STING (sense strand: 5′-GGAUCCGAAUGUUCAAUCAtt-3′ [SEQ ID NO. 2]), in the presence of Lipofectamine. siRNA transfection was performed for 24 hours, after incubation total RNA was isolated using the RNeasy® kit (Qiagen). cDNA was synthesized using High Capacity cDNA Reverse Transcription Kit (applied biosystems™), and knock down of each gene was measured by quantitative RT-PCR using specific primer/probe mouse STING (forward 5′-AACACCGGTCTAGGAAGCAG-3′ (SEQ ID NO. 3), reverse 5′-CATATTTGGAGCGGTGACCT-3′ (SEQ ID NO. 4) and probe 5′-CATCCAGC-3′) (SEQ ID NO. 5), mouse IRF-3 (forward 5′-CAAGAGGCTTGTGATGGTCA-3′ (SEQ ID NO. 6), reverse 5′-GCAAGTCCACGGTTTTCAGT-3′ (SEQ ID NO. 7) and probe 5′-AGGAGCTG-3′ (SEQ ID NO. 8)). The siRNA transfected cells were stimulated with tumor-derived DNA and amount of IFN-β was measured as described above. WT macrophages were cultured in 96-well plates at a density of 5×10⁴ cells per well and transfected with 10 nM siRNA targeting mouse cGAS (sense strand: 5′-GAUUUCUGCUCCUAAUGAAtt-3′ (SEQ ID NO. 9); antisense strand: 3′-UUCAUUAGGAGCAGAAAUCtt-5′ (SEQ ID NO. 10)), or scrambled siRNA in complex with Lipofectamine RNAiMAX. siRNA transfection was performed for 48 hours, then total RNA was isolated using the RNeasy® kit (Qiagen). cDNA was synthesized using High Capacity cDNA Reverse Transcription Kit (applied biosystems™), and knock down of cGAS was measured by quantitative RT-PCR using specific primer/probe sets (mouse cGAS—forward: 5′-GAA TCT TCC GGA GCA AAA TG-3′ (SEQ ID NO. 11), reverse: 3′-GGC AGT TTT CAC ATG GTA GGA-5′ (SEQ ID NO. 12) and probe: 5′-CATCCAGC-3′ (SEQ ID NO. 13)). The siRNA-transfected cells were stimulated with 20 or 200 ng of tumor-derived DNA per well. After 12 hours, supernatants were collected and the amount of IFN-β was assessed by ELISA (PBL Interferon Source). For IFN-β transcript assay, each tumor cells were injected into mice and CD45⁺ cells were collected by cell sorting. Q-PCR analysis was performed described above.

Dendritic cell cytokine and microarray analysis. BMDCs were generated from WT or STING^{-/-} mice as described above. After tumor-derived DNA stimulation for 7 hours, supernatants were collected and the amount of IL-6, IL-12p40, and TNF-α was measured by ELISA (eBioscience). Stimulated BMDCs with tumor-derived DNA were lysed and total RNA was isolated using RNeasy® kit (Qiagen). Isolated RNA was submitted for Affymetrix GeneChip analysis to the Functional Genomics Facility at the University of Chicago. The RNA integrity was assessed by Agilent 2100 Bioanalyzer (Agilent Technologies), and the concentration/purity of RNA was determined by NanoDrop 1000 (Thermo Scientific). All RNA samples used for microarray analysis had RNA Integrity Number>8.0, OD260/280 and OD260/230 ratio>1.8. The arrays (Affymetrix mouse genome 430 2.0_{—} were scanned by Affymetrix Gene Chip Scanner 3000 7G and CEL. Intensity files were generated by Gene Chip Operating Software v. 1.4 (MicroArray Suite 5.0). dChip software was used to analyze the microarray data. Using dChip software, the genes scored as “absent” or with signal intensity <100 were first filtered out. Fold-change of gene expression was calculated by dividing signal intensity value of genes of WT or STING^{-/-} tumor-derived DNA transfected BMDCs with that of media treated WT BMDCs.

Skin transplantation. Skin transplantation was performed as previously described (Molinero, et al., 2008). Briefly, full-thickness donor flank skin pieces (0.5-1 cm²) were positioned on a graft bed prepared on the flank of the recipient. The time point of rejection was defined as the complete necrosis of the graft.

PCR and quantitative RT-PCR analysis of IFN-β. Human melanoma 624 tumor cells were stained with DRAQ5 (Cell Signaling) and inoculated into mice subcutaneously. After overnight, tumor cells were isolated and single cell suspensions were prepared. After anti-mouse CD45-PE (30-F11), CD11c-Percp-Cy5.5 (N418) and anti-human HLA-A,B,C-AF 488 (W6/32) were used for staining. After gating live cells by DAPI staining, CD45-PE and CD11c-PerCP-Cy5.5 positive cells were collected by cell sorting with FACSAria III (BD) in the Flow Cytometry Core Facility in University of Chicago. Total DNA was isolated with All Prep® DNA/RNA Micro Kit (Qiagen) and DNA concentration was measured with ND-100 spectrophotometer (Nanodrop). PCR primers were designed with Primer-BLAST program (NCBI). PCR reaction cocktail was prepared using Maxima Hot Start PCR Master Mix (Thermo scientific) and performed using PTC-200 Peltier Thermal Cycler (MJ Research). PCR product was run on a 1.5% agarose gel and visualized with EtBr. Gel pictures were obtained using an ultraviolet transilluminator (Kodak). For RT-PCR analysis of IFN-β, B16.SIY melanoma cells were inoculated into mice (5 mice per group). Single cell suspensions were prepared described above and stained with antibodies anti-mouse CD45-PE (30-F11), anti-mouse CD11b-PacBlue (M1/70) and anti-mouse CD11c-PEcy7 (N418). Stained cells were collected by cell sorting with FACSAria III (BD). Total RNA was isolated using the RNeasy® kit (Qiagen). cDNA was synthesized using High Capacity cDNA Reverse Transcription Kit (applied biosystems™). Q-PCR reaction was performed using TagMan Gene Expression Master Mix (A&B) and 7300 Real Time PCR system (A&B).

ImageStream analysis. Harvested tumors were incubated with collagenase (50 unit/ml; Worthington Biochemical Corporation) for 2 hrs at 37° C. Single suspensions of tumor-derived cells were prepared by homogenization using a syringe plunger and cell strainer.

After antibody staining, single cell images were acquired with ImageStream^{x}Mark II (Amnis). Collected data were analyzed with IDEAS 5.0 software (Amnis). Single-stained control cells were used for compensation. Cells were gated for single cells with the area and aspect ratio and for focused cells with Gradient RMS feature. For DRAQ5 uptake assay, B16 melanoma cells were incubated with DRAQ5 (5 μM) for 15 minutes. After extensive washing with PBS, stained tumor cells were inoculated into mice subcutaneously. The next day, the tumor bump was harvested and tumor-derived cells were isolated and a single cell suspension was prepared described above. Cells were stained with LIVE/DEAD Fixable Dead cell stain Kits (Invitrogen), anti-mouse-CD45-PECy5 (30-F11), and CD11c-PECy7 (N418), followed by analysis with the ImageStream^{x}MarkII (Amnis). For the Edu experiment, B16 melanoma or 1969 sarcoma cells were incubated with Edu (10 μM) for overnight in complete DMEM culture medium. After extensive washing, tumor cells were stained with DRAQ5 or CellTracker™Green CMFDA (Invitrogen) and inoculated into mice. The next day, the tumor bump was harvested, made into a single cell suspension as above, and stained with anti-mouse CD45-PECy5 and CD11c-PECy7. Edu detection (either Alexa Fluor 555 or Alexa Fluor 647) was performed using Click-iT® EdU Imaging Kits (Invitrogen). Non-labeled tumor cells were used as a negative control through the same staining procedure. For pIRF3 staining, tumor single cell suspensions were stained with LIVE/DEAD Fixable Dead cell stain, anti-mouse CD45-PECy5, CD11c-PECy7 and permeabilized with Foxp3 Fixation/Permeabilization kit (eBioscience). After blocking with Normal Mouse Serum, cells were stained with pIRF3 antibody (Cell Signaling, Cat # 4947) and subsequently were stained with anti-rabbit IgG-PE secondary antibody (Invitrogen). For nuclear staining, stained cells were incubated with NucBlue™Fixed Cell Stain (Invitrogen) for 5 minutes. For pTBK1 staining, the same procedure was used as above except using a pTBK1-specific antibody (cell signaling, Cat # 5483).

Statistical analysis. The student's t-test was used for statistical analysis. P values of less than <0.05 were considered statistically significant.

### Example 2

### STING and IRF3 are Required for Spontaneous T Cell Activation Against Tumors In Vivo.

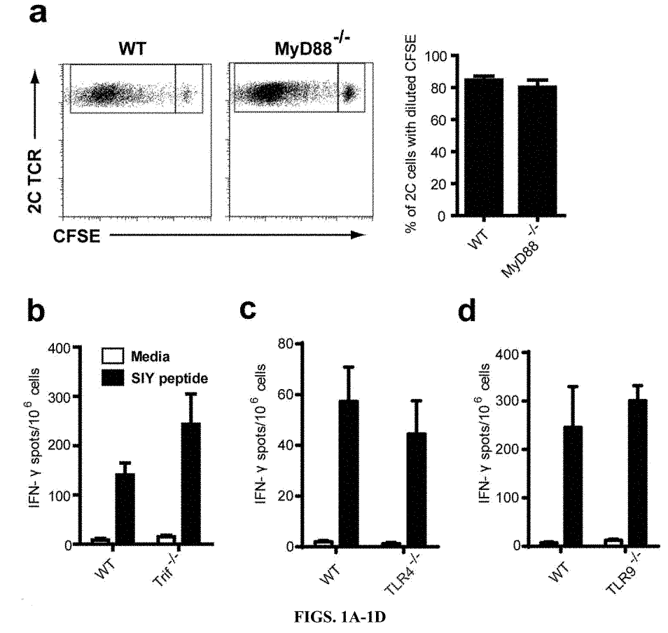

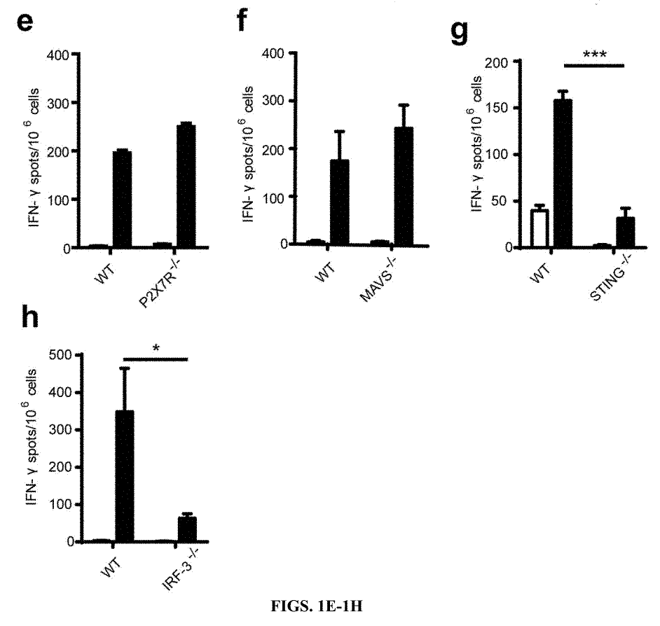

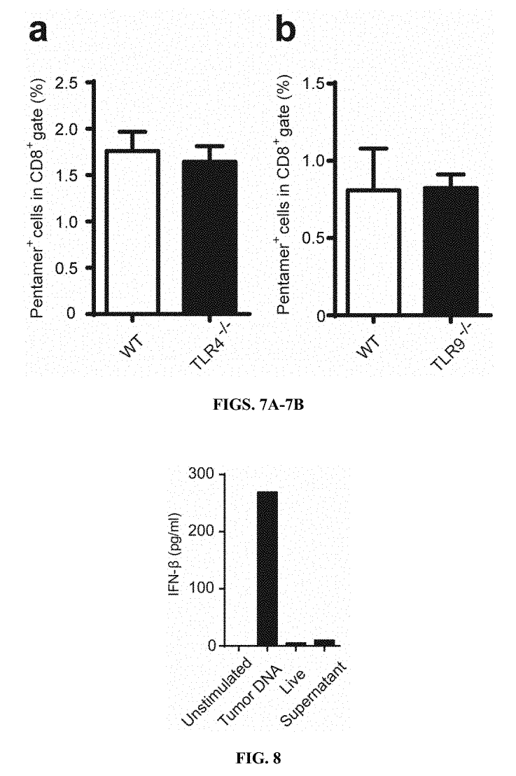

The inventors pursued a working model in which innate immune sensing pathways might detect tumor-derived factors, induce type I IFN production, and lead to cross-priming of tumor antigen-specific CD8⁺ T cells in the host (Fuertes, et al., 2011; Diamond, et al., 2011). To begin to address host requirements for a natural anti-tumor T cell response, gene-targeted mice deficient in specific pathways were utilized. To determine whether host Toll-like Receptor (TLR) pathways were required for spontaneous CD8⁺ T cell priming, the inventors utilized MyD88^{-/-} or TRIF^{-/-} mice. Because MyD88 can function in a T cell-intrinsic fashion (Zhou, et al., 2009), the inventors performed adoptive transfer of wildtype CFSE-labeled 2C TCR Tg T cells (that are specific for the model antigen SIY) into WT or MyD88^{-/-} mice and challenged with B16.SIY tumors (Zhou, et al., 2005). No defect in T cell proliferation or accumulation of divided cells was observed in MyD88^{-/-} mice (FIG. 1a). Similarly, endogenous CD8⁺ T cell priming against tumor-derived SIY was intact in TRIF^{-/-} mice (FIG. 1b), suggesting that the TLR system is not mandatory for spontaneous priming of anti-tumor CD8⁺ T cells in vivo (Stetson, et al., 2006; Ishii, et al., 2006). The inventors also examined CD8⁺ T cell responses in mice specifically lacking TLR4 or TLR9, and no defect was observed using either IFN-γ ELISPOT (FIG. 1c,d) or SIY peptide/K^{b} pentamer staining (FIG. 7). A second candidate mechanism of innate immune sensing is through extracellular ATP, as it has been suggested that dying tumor cells might release ATP which could be sensed by P2X7R on APCs (Ghiringhelli, et al., 2009). However, the inventors found no defect in spontaneous priming of CD8⁺ T cells against tumor-associated antigens in P2X7R^{-/-} mice (FIG. 1e). The inventors also examined a role for the defined RNA sensing pathway using MAVS^{-/-} mice which lack the critical adapter molecule for RIG-I- and MDA5-dependent innate immune activation. However, no defect of CD8⁺ T cell priming in MAVS^{-/-} mice was observed (FIG. 1f).

The inventors therefore turned to the other remaining defined pathway for innate immune sensing that can lead to type I IFN production, which is cytosolic DNA sensing via the STING pathway. Recent studies of pathogen sensing have identified a pathway involving an endoplasmic reticulum resident protein called STING leading to IRF3 activation and IFN-β transcription (Ishikawa, et al., 2009). STING has been shown to function as an adapter molecule for DNA recognition pathways, with recent data suggesting that this occurs indirectly through binding of cyclic dinucleotides, which can be generated from metabolized DNA via the enzyme cGAS (Wu, et al., 2013; Abe, et al., 2013; Burdette, et al., 2011). Using both STING^{-/-} and IRF3^{-/-} mice, the inventors observed a substantially diminished CD8⁺ T cell response against tumor-associated antigen in vivo (FIG. 1g, h). These data indicate that STING and IRF3 in host cells are required for spontaneous CD8⁺ T cell priming response against tumors.

### Example 3

### Tumor-derived DNA Induces IGN-B Production by STING and IRF-3 Dependent Pathways

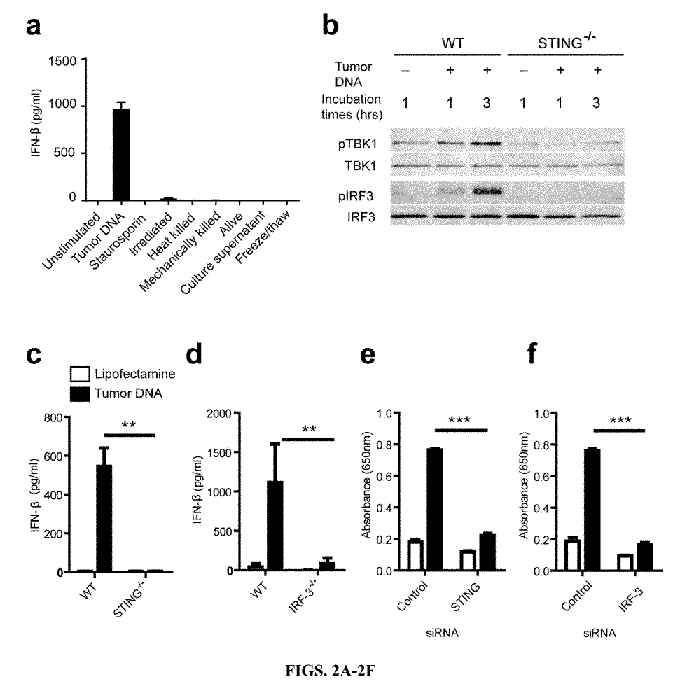

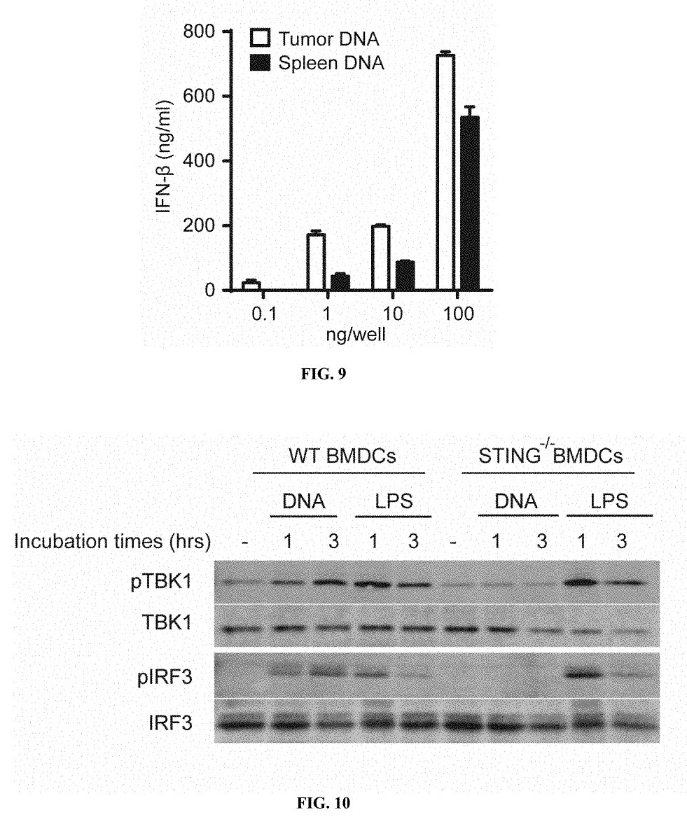

The inventors turned to an in vitro system to screen fractions of B16 tumor cell extracts and tumor cells killed using a variety of approaches, to determine which preparation might be capable of inducing IFN-β from DCs. Tumor cells killed in multiple ways, including by mechanical disruption, or supernatants from spent B16 cultures failed to induce IFN-β production by bone marrow-derived DCs (FIG. 2a). Based on recent reports characterizing a cytosolic DNA sensing pathway that can detect intracellular viruses, bacteria, and Plasmodium falciparum and drive type I IFN production (Unterholzner, et al., 2010; Takaoka, et al., 2007; Sharma, et al., 2011; Henry, et al., 2007), the inventors examined whether tumor-derived DNA might act similarly. Indeed, B16 melanoma-derived total DNA combined with Lipofectamine provoked IFN-β production by DCs (FIG. 2a). The inclusion of Lipofectamine was necessary, suggesting that the DNA needed to gain entry to the cytosol. Treatment of the tumor-derived DNA preparation with DNAse I abolished this stimulatory effect, supporting the contention that it is DNA in this preparation which is functional (data not shown). In contrast, tumor-derived RNA was minimally stimulatory (data not shown). In immortalized macrophages cells, tumor-derived DNA in combination with Lipofectamine also induced production of IFN-β (FIG. 8). In addition to tumor-derived DNA, normal cell-derived DNA isolated from splenocytes also induced production of IFN-β in mouse BMDCs when combined with Lipofectamine in vitro (FIG. 9), suggesting that there is unlikely to be a unique property of DNA derived from transformed cells that make it more stimulatory. Rather, there must be some characteristic of the tumor cell context that favors DNA transfer to host APCs as tumors become established in vivo.

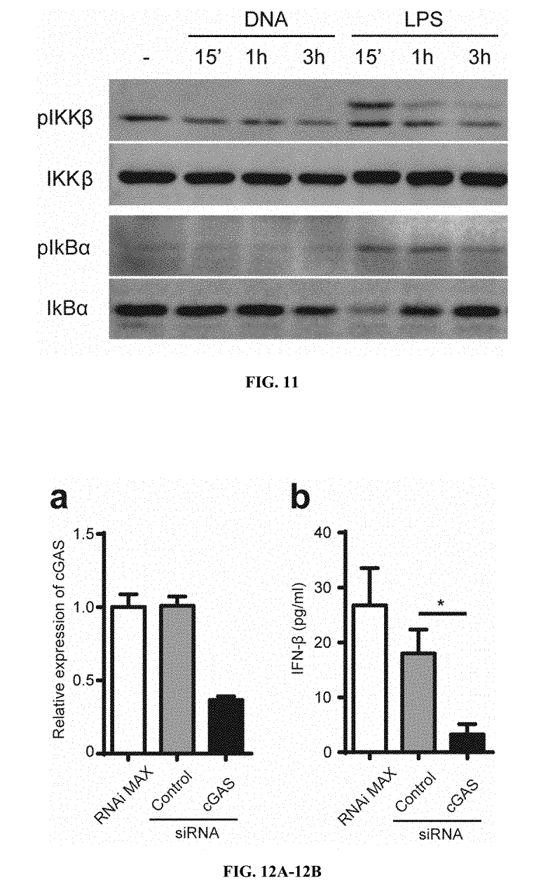

To assess activation of the STING pathway, the inventors performed Western blot analysis to assess phosphorylation of TBK1 and IRF-3 after tumor-derived DNA stimulation of bone marrow-derived DCs from WT or STING^{-/-} mice. The inventors indeed observed increased phosphorylation of TBK1 and IRF3 in DCs from WT mice which was not seen in DCs from STING^{-/-} mice. The amount of each protein was normalized with GAPDH loading control and the ratio of phosphorylated to total proteins was quantified (pTBK1/TBK1: WT (2.076) vs STING^{-/-}(0.705), pIRF3/IRF3: WT (0.308) vs STING^{-/-}(0.009); p<0.051, p<0.0001) (FIG. 2b). This amount of phosphorylation of TBK1 and IRF3 was comparable to what the inventors observed with LPS stimulation, although the phosphorylation of TBK1 and IRF3 with LPS stimulation was preserved in STING^{-/-} DCs (FIG. 10). In parallel, the inventors measured phosphorylation of IKKβ and IκBα after DNA stimulation as an indication of NFκB pathway activation. However, only minimal induction of IKKβ and IKBα phosphorylation was observed compared to LPS stimulation (FIG. 11), indicating that NFκB pathway activation is not a major component of the APC activation pathway induced by tumor-derived DNA.

To confirm whether the STING pathway was necessary for DC activation by tumor-derived DNA, the inventors stimulated bone marrow-derived DCs derived from WT, STING^{-/-} or IRF3⁻⁻ mice with B16-derived DNA and measured IFN-β production. Indeed, IFN-β production was severely blunted with STING^{-/-} or IRF3^{-/-} DCs (FIG. 2c, d). As a confirmatory approach, the inventors utilized a reporter cell line expressing the Secreted Embryonic Alkaline Phosphatase (SEAP) enzyme driven by the IFN-β-inducible ISG54 promoter. In this system, specific siRNAs for STING or IRF3 resulted in substantial inhibition of IFN-β-inducible ISG54 promoter activity after stimulation with tumor-derived DNA (FIG. 2e,f). These data indicate that tumor-derived DNA can induce production of IFN-β when introduced into APCs in vitro, via a mechanism dependent upon STING and IRF3.

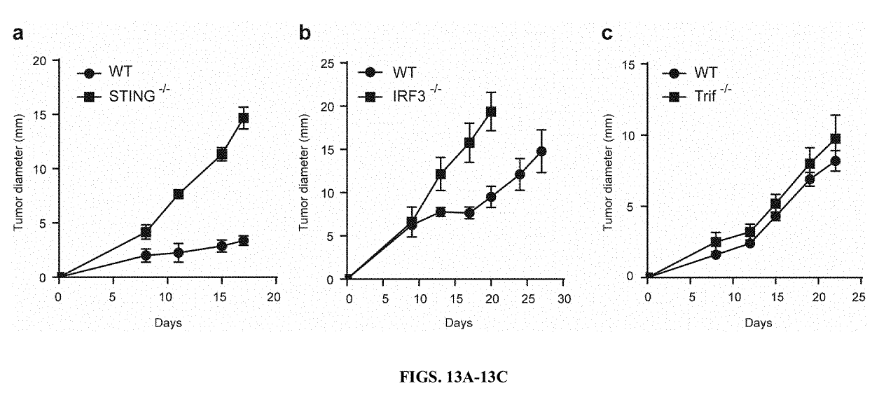

Recent data have indicated that the ultimate direct ligand of STING is cyclic dinucleotides, generated from DNA following metabolism by the enzyme cGAS (Wu, et al., 2013; Sun, et al., 2013). To assess whether the activation of the STING pathway by tumor-derived DNA also was occurring through this mechanism, the inventors utilized siRNA knockdown of cGAS in macrophages in vitro. In fact, markedly decreased production of IFN-β in response to tumor DNA stimulation was observed when cGAS levels were reduced (FIG. 12), suggesting that tumor DNA introduced into the cytosol of APCs activated the STING pathway in a cGAS-dependent fashion.

### Example 4

### Sting^{-/-} Mice show Defective Tumor Control and fail to Sustain T Cell Expansion

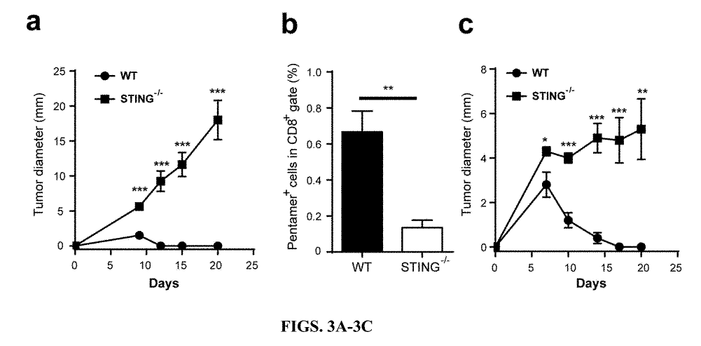

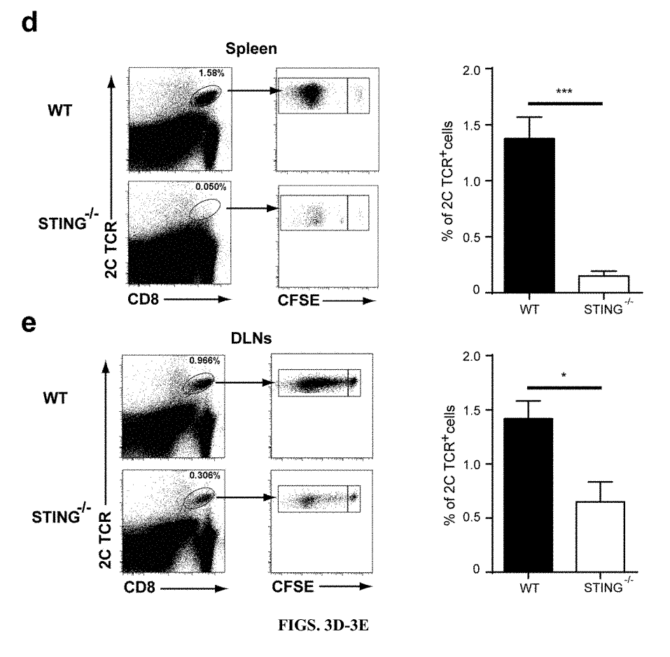

To determine effects of the host STING pathway on tumor growth control, the inventors utilized several model systems. First, B16 melanoma grows more slowly in immune-competent C57BL/6 mice than in immune-deficient RAG^{-/-} mice, suggesting that there is a modest effect of host immunity mediating partial tumor control. The inventors therefore measured the rate of tumor growth in syngeneic wildtype, STING^{-/-}, and IRF3^{-/-} mice. As expected, tumor growth was more rapid in STING^{-/-} and IRF3^{-/-} mice. In contrast, tumor growth was not altered in Trif^{-/-} mice, consistent with a lack of apparent necessity of TLR pathways in spontaneous priming of anti-tumor T cells (FIG. 13). The inventors also explored conditions in which immunogenic tumors are normally spontaneously rejected completely. As one approach, B16.SIY tumors were implanted into WT or STING^{-/-} mice on a 129 genetic background, which allows tumor rejection likely due to minor histocompatibility antigen differences in addition to tumor-specific antigens. In contrast to rejection in WT mice, tumors grew progressively in the absence of host STING (FIG. 3a). SIY peptide-specific CD8⁺ T cell responses also were significantly decreased in STING^{-/-} mice (FIG. 3b) in this system. In order to utilize a system that was completely syngeneic, the inventors utilized the immunogenic tumor called 1969, that was induced by treating immune-deficient mice with methylcholanthrene in the C57BL/6 background. This tumor as well was rejected in wildtype mice but failed to be rejected in STING^{-/-} mice (FIG. 3c). Collectively, these data suggest that, like endogenous T cell priming, immune-mediated tumor control requires the host STING pathway.

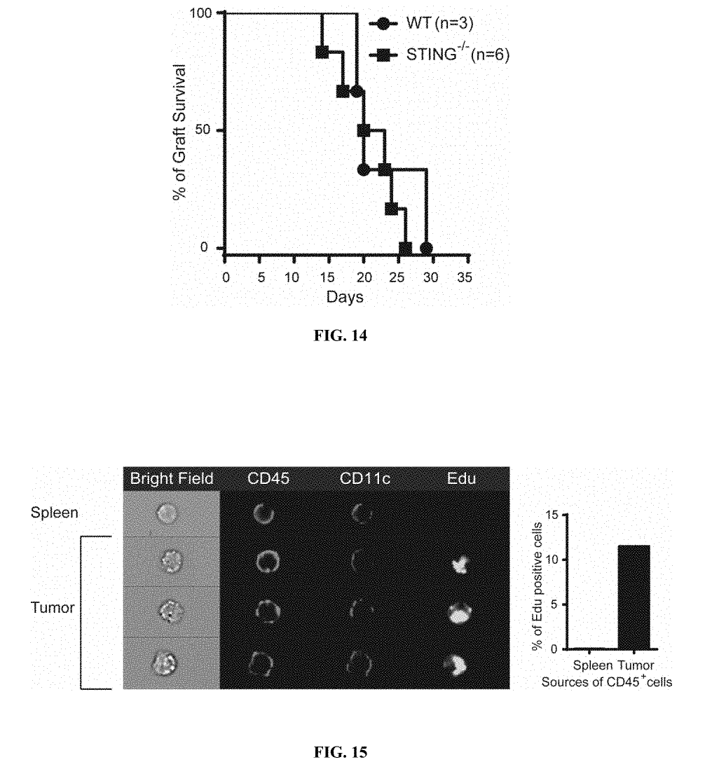

The inventors were concerned that STING^{-/-} mice might display a more global immune deficiency than what would be expected based solely via an effect on cytosolic DNA sensing. To this end, the inventors investigated skin graft rejection across minor histocompatibility antigen differences. Skin was transplanted from male STING^{-/-} donors into female STING^{-/-} recipients, and the rate of rejection was comparable to that seen with wildtype donor and recipient pairs (FIG. 14). These data indicate that not all tissue-based T cell rejection processes are defective in STING^{-/-} mice, and argue that the tumor cell context has special properties that render host T cell priming dependent on the STING pathway.

To evaluate in more detail the mechanism by which absence of host STING resulted in impaired anti-tumor T cell responses in vivo, the inventors adoptively transferred CFSE-labeled 2C TCR Tg T cells into WT and STING^{-/-} mice and measured T cell proliferation by CFSE dilution after B16.SIY challenge. Interestingly, a similar number of cell divisions was observed in both recipients, but the CFSE-diluted 2C cells failed to accumulate in STING^{-/-} mice, in the spleen and lymph nodes (FIG. 3d). This is a pattern that has been seen in other models of poor T cell costimulation leading to non-productive T cell activation, and suggests that the STING pathway might be required not only for IFN-β production but additionally for expression of other T cell costimulatory factors (Abe, et al., 2013; Hoebe, et al., 2003). To evaluate a potentially broader DC activation property of DNA, DCs were generated from WT or STING^{-/-} mice and stimulated with tumor-derived DNA, and gene expression profiling was performed. In fact, tumor-derived DNA induced expression of a broad spectrum of genes encoding multiple critical cofactors for T cell activation, including cytokines (e.g. IL-12), chemokines (e.g. CXCL9) and costimulatory molecules (e.g. CD40; FIG. 4a). These were induced in WT but not STING^{-/-} DCs. ELISA confirmed STING-dependent induction of IL-6, TNF-α, and IL-12 (FIG. 4b-d) by tumor DNA. Induction of these factors by DNA was intact in bone marrowderived DCs from MyD88 and Trif knock-out mice, supporting a TLR-independent mechanism of this DC activation (data not shown). The inventors speculate that induction of some of these genes might not be directly induced via the STING pathway but rather in response to the secreted type I IFNs induced.

### Example 5

### Tumor-derived DNA is Transferred to host APCS In Vivo

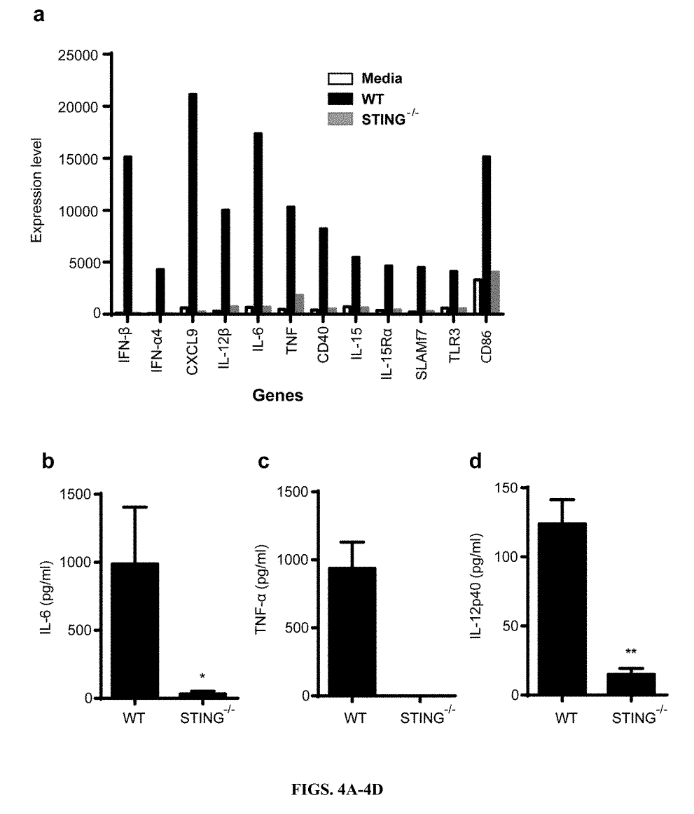

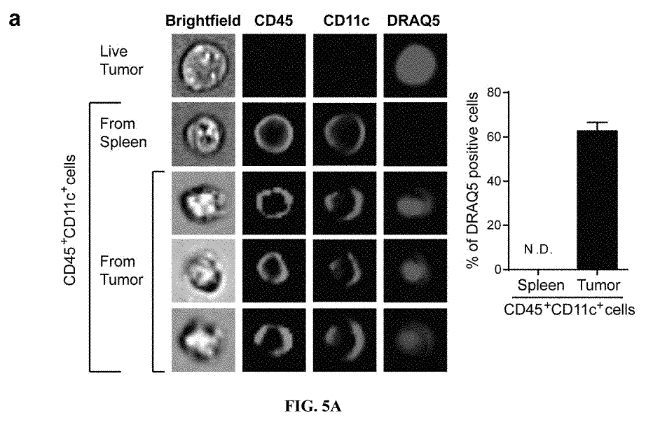

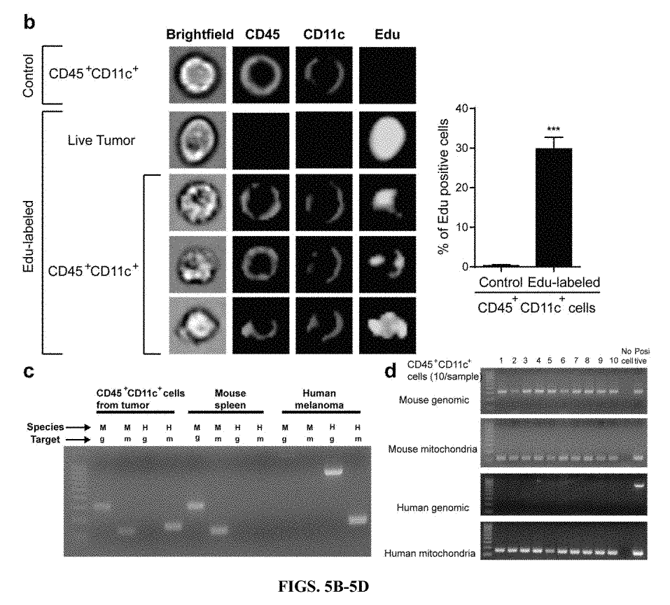

If DNA is the relevant tumor-derived material initiating engagement of the STING pathway in vivo, then it should be possible to detect tumor-derived DNA within host APCs in the tumor microenvironment. This possibility was investigated using three complementary approaches. As a first approach, the inventors stained tumor cells in vitro with DNA-intercalating dye DRAQ5 and then implanted these tumor cells in vivo. In order to avoid dilution of the dye as a consequence of cell proliferation, the inventors analyzed host inflammatory cells one day after tumor injection. The early tumor bump was harvested, disrupted into a single cell suspension, and then analyzed by cytometry. In order to ensure that the analysis focused exclusively on host myeloid cells and not fusion heterokaryons or cell aggregates, single cell analysis using the Amnis ImageStream instrument was employed. Host DCs were analyzed based on staining for CD11c and CD45. Indeed, approximately 60% of CD45⁺CD11c⁺ cells showed positive staining with tumor cell-derived DRAQ5, in a diffuse staining pattern (FIG. 5a). In the same single cell suspension, tumor cells were negative for CD45 and CD11c staining but positive for DRAQ5. This DRAQ5 staining was not seen in normal spleen cells, and was observed in only a small population of splenocytes obtained from the tumor-injected mice (FIG. 5a). Using an in vitro transwell system, the inventors found that DRAQ5 transfer was not detected in non-labeled cells separated by a membrane, arguing that detection in DCs was not a consequence of leaking of the DRAQ5 dye from the tumor cells (data not shown).

As a second approach, the inventors labeled tumor cells with the nucleotide analogue EdU prior to injection into mice, and utilized ImageStream for single cell analysis for EdU staining. Non-labeled tumor cells were used as a negative control. Similar to DRAQ5, the inventors observed EdU staining on a large population of tumor-infiltrating CD45⁺ CD11c⁺ cells (FIG. 5b). The inventors also observed tumor-derived DNA transfer to host APCS using the 1969 tumor cell system, arguing that this phenomenon is not unique to B16 melanoma (FIG. 15). Compared to DRAQ5 staining, the percentage of EdU-positive cells in host APCs was consistently lower than that of DRAQ5-positive cells. This difference could be because only a subset of the tumor DNA incorporates EdU with this strategy.

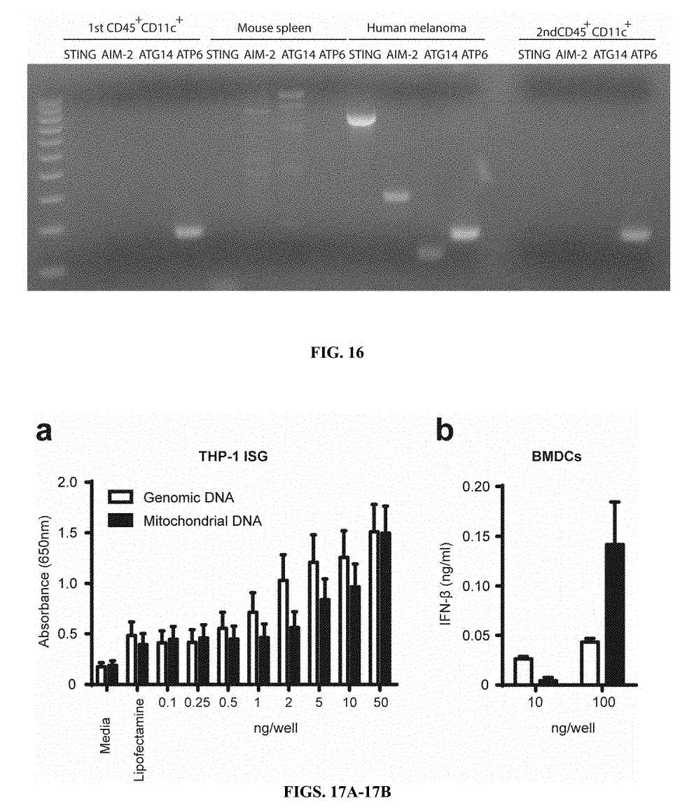

As a third approach, the inventors utilized a human xenograft model which enabled the use of species-specific PCR to interrogate host DCs for the presence of tumor-derived DNA. This approach also allowed evaluation of whether genomic DNA or mitochondrial DNA was predominantly detected. The human melanoma cell line 624 was implanted subcutaneously, and tumor-infiltrating CD45⁺ cells were isolated one day later by flow cytometric sorting. To ensure high purity, negative sorting was done on human HLA⁺ cells and positive sorting on cells expressing murine CD45 and CD11c. Re-analysis of 10,000 sorted cells revealed no detectable human melanoma cells (data not shown). PCR was then performed using primers specific for human mitochondrial DNA (ATP synthase 6) and genomic DNA (TMEM 173), and also for mouse sequences (ifi204 and ATP synthase 6) as a control. Using this method, the inventors indeed detected human mitochondrial DNA sequences in sorted mouse APCs (FIG. 5c). However, genomic DNA was not detected using this approach. The inventors similarly failed to detect the presence of two other human genomic DNA sequences by PCR (AIM2 and ATG14, FIG. 16) in these highly purified host APCs. Because of the remote possibility that one contaminating tumor cell among 10,000 host APCs might be detected, the inventors increased the stringency of this assay by doing limiting dilution of the sorted APCs down to 10 cells per well for PCR. Using this technique as well, mitochondrial DNA was detected in all samples (FIG. 5d), arguing that it is indeed present within the host APCs and is not due to contamination by human tumor cells. While genomic DNA was not detected, the inventors cannot completely rule out its transfer because partial degradation may occur in host APCs. To assess whether mitochondrial DNA was itself capable of inducing type I IFN production, the inventors separately purified mitochondrial and genomic DNA from B16 melanoma cells and found that they both stimulated type I interferon production when introduced into THP-1 ISG reporter cells and BMDCs (FIG. 17). Taken together, these data suggest that tumor-derived DNA, at least from mitochondrial sources, can be detected in host APCs early following tumor implantation in vivo and could be sufficient for activation of the STING pathway.

### Example 6

### Tumor-infiltrating host Apcs Produce IFN-β via a Sting-dependent Mechanism In Vivo

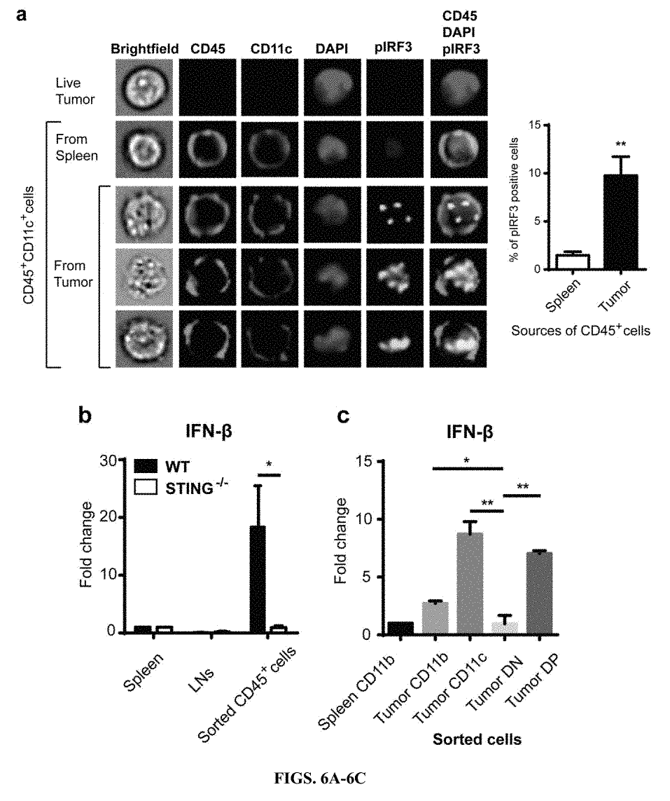

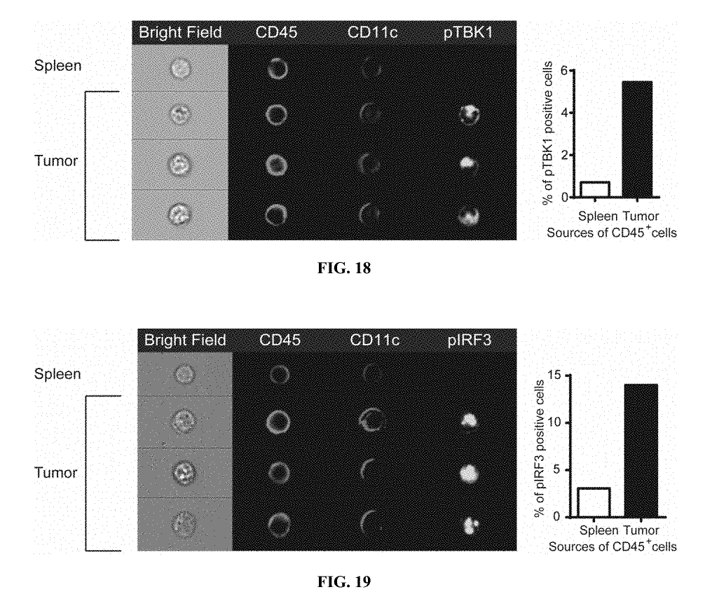

In as much as DNA transfer to host DCs appeared to occur rapidly in vivo, the inventors investigated whether those host APCs could activate the STING pathway and produce IFN-β within the same time frame. To this end, B16 melanoma cells were implanted subcutaneously, and one day later the tumor-infiltrating CD45⁺ cells were analyzed for phopho-IRF3 induction by ImageStream. As shown in FIG. 6a, despite this being a snapshot in time at an early time point, approximately 10% of tumor-infiltrating CD45⁺ cells showed pIRF3 staining which appeared to be translocated to nucleus. As a control, in the same single cell suspension, tumor cells were negative for CD45, CD11c and pIRF3 staining. CD45⁺ cells in the spleen also showed minimal staining for pIRF3 (FIG. 6a). In parallel, activation of the upstream kinase TBK1 was similarly assessed by phosphorylation status. Similarly to pIRF3, pTBK1 was detected in a subset of CD11c⁺cells from the tumor microenvironment ex vivo (FIG. 18). The inventors were concerned that perhaps the early time points being examined might not reflect the status of a stable tumor microenvironment in a palpable tumor. Therefore, the inventors also examined pIRF3 staining in CD11c⁺ cells in 7-day established B16 melanoma. Similarly to the early time points, pIRF3 staining in CD11c⁺ cells was also observed in these larger established tumors (FIG. 19).

To assess whether IFN-β was produced by the early tumor-infiltrating APCs, the inventors isolated tumor-infiltrating CD45⁺ cells from WT or STING^{-/-} mice after injection of B16.SIY melanoma by flow cytometric sorting, then performed qRT-PCR. A significant induction of IFN-β transcripts was observed in CD45⁺ cells from WT mice but not from STING^{-/-} mice (FIG. 6b). Further investigation of the subpopulations that produced IFN-β was pursued by flow cytometric sorting, and revealed that CD11c single positive or CD11 c/CD11b double positive cells appeared to be major source of IFN-β production, whereas CD11b single positive cells were not major producers (FIG. 6c). These combined data suggest that innate immune sensing of tumors can induce phosphorylation of TBK1 and IRF3 and lead to production of IFN-β via a STING-dependent pathway by host DCs in the tumor microenvironment in vivo.

### Example 7

DMXAA promotes STING aggregation at peri-nuclear sites. In this first experiment the inventors used the ImageStream, a cytometer and microscope that permits analysis of single cells, to study the activation of STING. The inventors saw a disperse pattern outside the nucleus. Only 15 min after the addition of DMXAA, STING aggregated in perinuclear sites. The inventors were able to quantify the activation of STING using the software of the ImageStream, and only 15 min was necessary to determine that around 70% of cells present these aggregates.

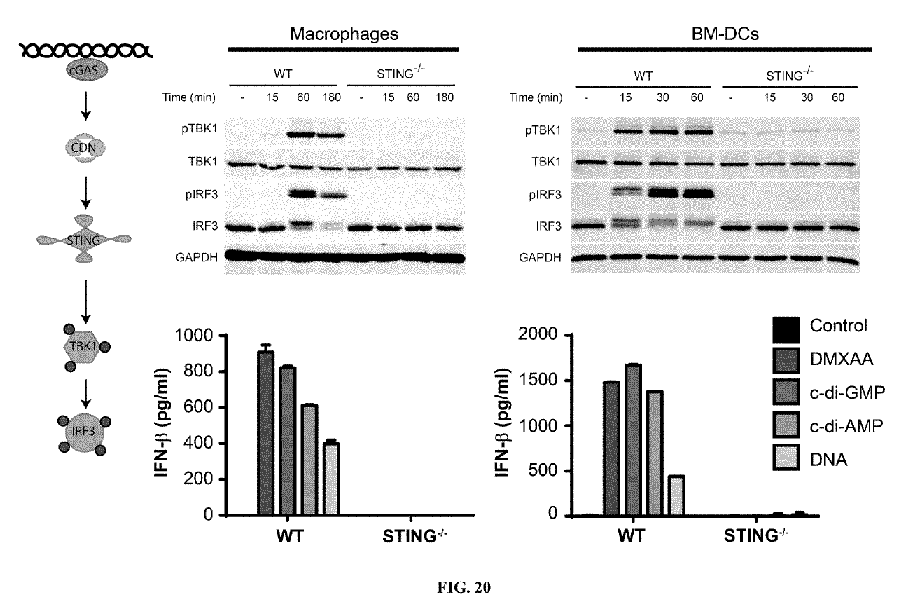

DMXAA activates the STING pathway and triggers type I IFN production. The inventors also checked the pathway downstream STING aggregation by assessing the phosphorilation of TBK1 and IRF3 and the production of IFN-β in WT and STING macrophages. The inventors observed a rapid and potent phosphorilation of TBK1 and IRF3 in WT cells, but not in STING deficient cells, which lead to a high production of IFN-β only in WT macrophages. The amount of IFN-β produced was similar as the amount produced after stimulation with cyclic dinucleotides and higger than the amount produced by stimulation with DNA. Using BM-DC from WT or STING ko mice, the inventors observed the same potent activation of the pathway and a high production of IFN-β. See FIG. 20.

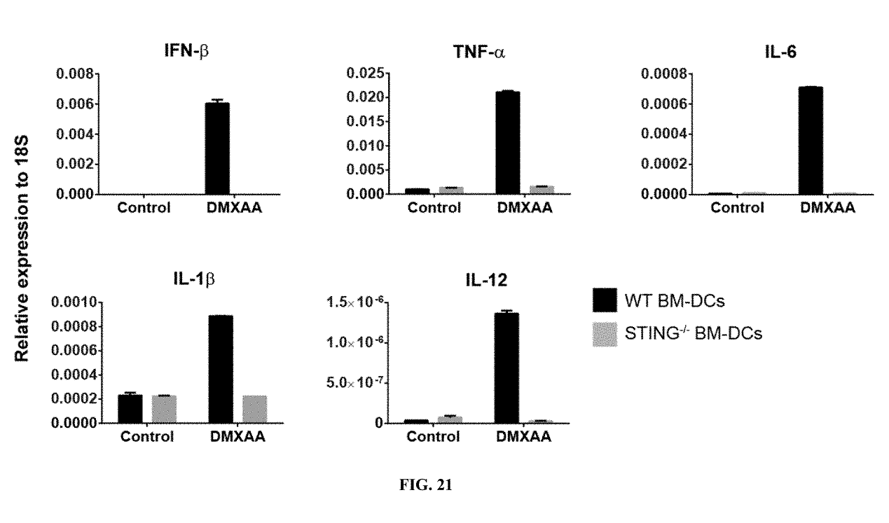

Induction of cytokines in BM-DC by DMXAA is STING-dependent. As the WT APCs showed a high activation of the STING pathway after addition of DMXAA, the inventors wanted to confirm if those cells were activated. Apart from IFN-β, BM-DCs also upregulated other cytokines such as TNFα, IL6, IL1, IL10, and IL12 in a STING dependent manner. See FIG. 21.

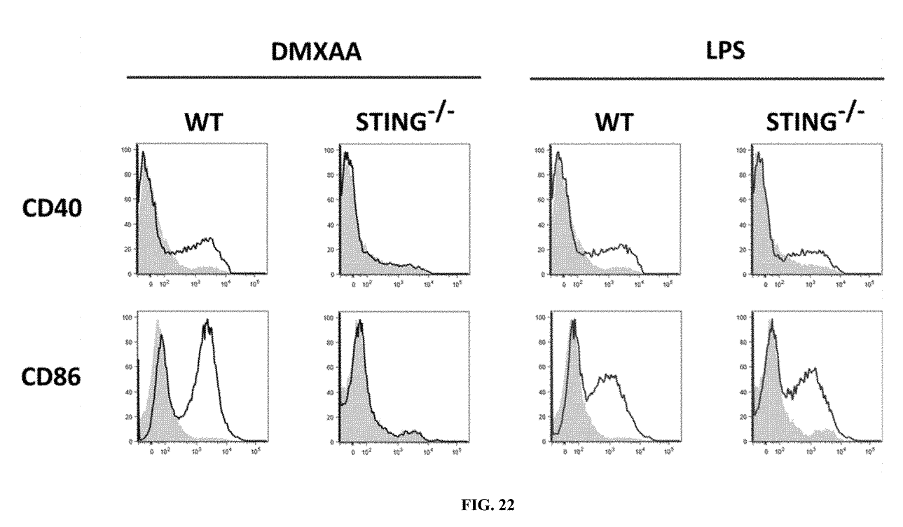

Induction of costimulatory ligands in BM-DC by DMXAA is STING-dependent. In addition, WT DCs upregulated activation markers such as CD40 and C86 in a STING dependent manner. The STING deficient cells were stimulated with LPS to demonstrate functionality, and in this case there was no difference with the WT cells. See FIG. 22.

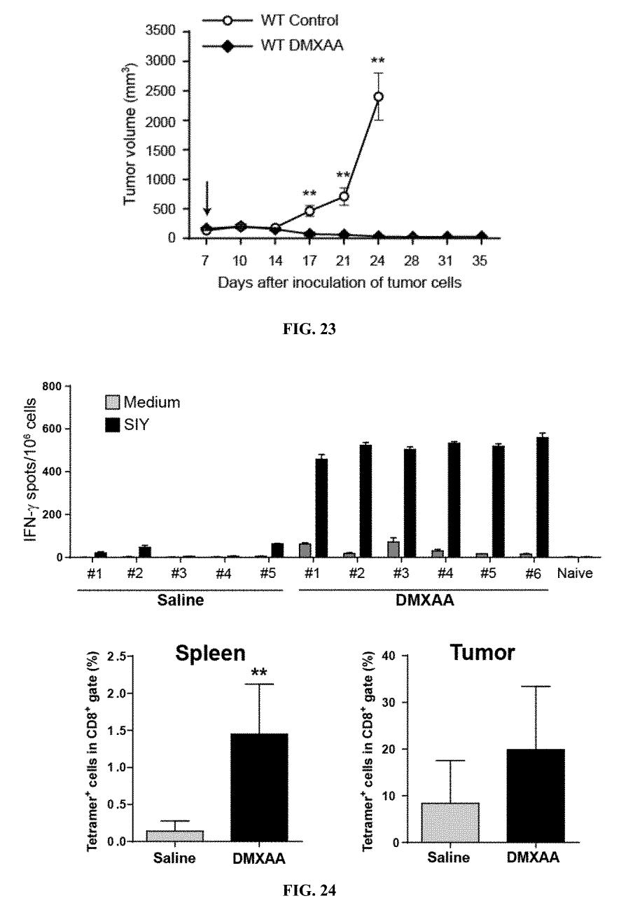

Intratumoral DMXAA triggers rejection of B16.SIY tumors in WT mice. To determine whether DMXAA will rise a potent immune response in a mouse model of melanoma, the inventors injected the B16 melanoma cell line that overexpress the SIY peptide in the flank of B6 mice. And after one week, when tumors are around 100-200 mm3 in volume, the inventors treated those mice with a single dose intratumorally of DMXAA or saline and measure the tumor growth. Most of the mice treated with DMXAA (80-90%) reject the tumors. See FIG. 23. Similar results were obtained in trials with the human molecule.

DMXAA triggers a potent CD8⁺ T cell response against the tumor-expressed SIY antigen. The inventors also measured the specific response against the SIY antigen one week after the injection of DMXAA. The number of specific T cells that produce IFN-g upon stimulation with SIY was measured using an IFN-g ELISPOT, and a 10 fold increase in the DMXAA treated animals was observed. In addition, using a pentamer staining of SIY, the inventors observed a higher amount of CD8+ SIY specific T cells in the spleens and within the tumors of DMXAA treatted animals. See FIG. 24.

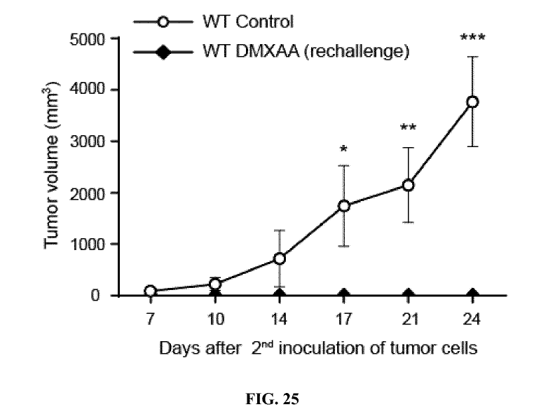

DMXAA protects animals against a second tumor rechallenge. Of all the animals in the DMXAA group that rejected the tumors, the majority of them did not grow any tumors when they were rechallenged with the same tumor cell line, which implies that they had generated immunologic memory. See FIG. 25.

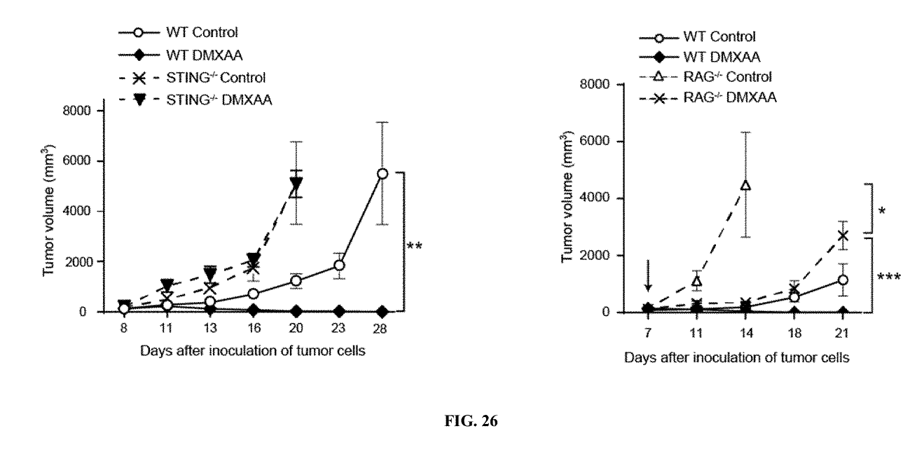

Failure of DMXAA to control tumor growth in STING^{-/-} and RAG^{-/-} mice. Finally, the inventors asked if DMXAA had any effect in STINGko and RAGko animal. As the inventors expected, DMXAA had no effect at all in animals deficient in STING, and DMXAA had a partial effect in RAGko mice. This indicates that alternative mechanisms other than the activation of T cells are implicated in the therapeutic effect of DMXAA. See FIG. 26.

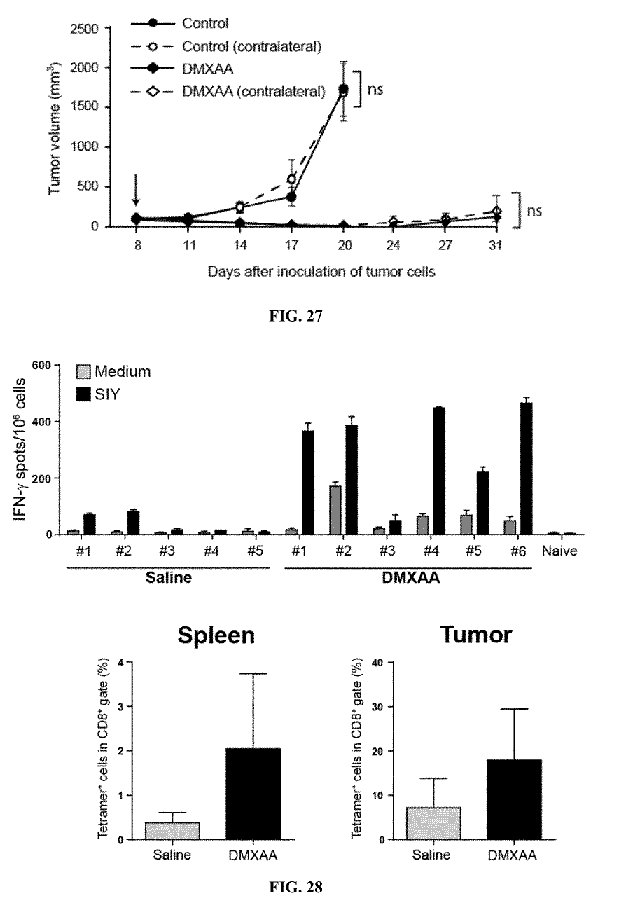

DMXAA triggers rejection of B16.SIY tumors in WT mice. See FIG. 27.

DMXAA triggers a potent immune response against SIY antigen. One week after

DMXAA injection, the inventors measured the endogenous T cell response against SIY by IFN-g ELISPOT and by assessing the CD8 SIY positive cells within the spleen and the tumors. The T cell response was highly increased in DMXAA treated animals. See FIG. 28.

### Example 8

### Brief Proposal to Study Cyclic Dinucleotides from Aduro as a Cancer Immunotherapy Strategy in Mouse Tumor Models

Animal tumor model and in vivo injection: B6 WT mice from 8 to 10 weeks of age (from Jackson) are injected subcutaneously in the right flank with 1×10⁶ B16.SIY.dsRed cells in 100 μL PBS. After one week of the injection, tumors are measured with calipers and volumes are calculated using the formula [length×(width)2]/2. When tumors are around 100 to 200 mm3 in size mice are treated intratumorally with a single dose of 25 micrograms per gram of body weight of DMXAA resuspended in 7.5% sodium bicarbonate. Control animals are treated with a single injection of 7.5% sodium bicarbonate (saline). As a comparison, the cyclic dinucleotide compounds from Aduro will be injected into tumors in parallel sets of mice. Tumor volumes are estimated twice a week using the formula described above.

Preparation of DMXAA stock for intratumoral injection: DMXAA (Vadimezan) is purchased from Selleckchem in a powder form. Upon arrival, DMXAA is resuspended in 7.5% of sodium bicarbonate to a final concentration of 6.25 mg/ml, and stored at −20° C. protected from light.

Measurement of the Immune Response against SIY antigen: After 7 days of the treatment of mice with DMXAA or saline, animals are sacrifice with CO2 and spleens extracted for analyzing the production of IFN-γ by splenocytes. The mouse IFN-γ enzyme-linked Immunospot assay (ELISPOT) from BD is used according to the manufacturer's protocol. In brief, splenocytes are plated at 106 cells/well and stimulated overnight with SIY peptide (160 nM), PMA (50 ng/ml) and ionomycin (0.5 μM) as positive control, or medium (DMEM supplemented with 10% heat-inactivated FCS, penicillin, streptomycin, L-arginine, L-glutamine, folic acid, and L-asparagine) as negative control. IFN-γ spots are detected using biotinylated antibody and avidin-peroxidase and developed using AEC substrate (BD Bioscience). Plates are read in an Immunospot Series 3 Analyzer and analyzed with ImmunoSpot software (Cellular Technology Ltd).

Tetramer staining of splenocytes and tumor infiltrate: After 7 days of the treatment of mice with DMXAA or cyclic dinucleotides, splenocytes and tumor infiltrate will be analyzed for SIY-specific CD8+ T cells detected by SIY/Kb pentamer staining. 5×10⁶ cells/sample are labeled with PE-MHC class I tetramers (Beckman Coulter or Proimmune) consisting of murine H-2Kb complexed to either SIYRYYGL (SIY) peptide or SIINFEKL (OVA) peptide as a negative control, anti-TCRβ-AF700 (clone H57-597), antiCD8-PO (clone 5H10), anti-CD4-PB (clone RM4-5), anti CD62L-PE_Cy7 (clone MEL-14), anti-CD44-APC (clone IM7) and the Fixable Viability dye eFluor780 (eBioScience). FACS analysis is performed using FACSCanto or LSR II cytometers with FACSDiva software (BD). Data analysis is conducted with FlowJo software (Tree Star).

### Example 9

### Direct Sctivation of STING in the Tumor Microenvironment Leads to Potent and Systemic Tumor Regression and Immunity

### Results

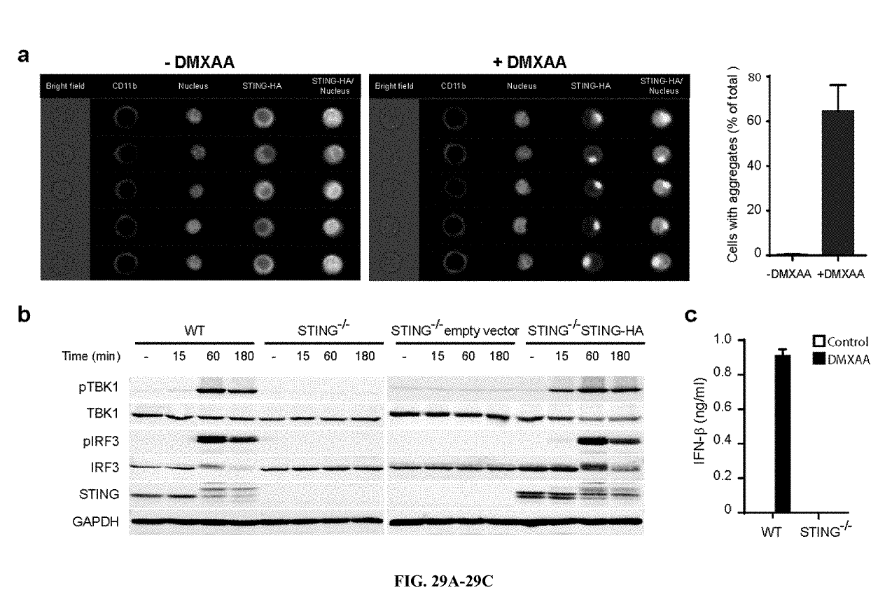

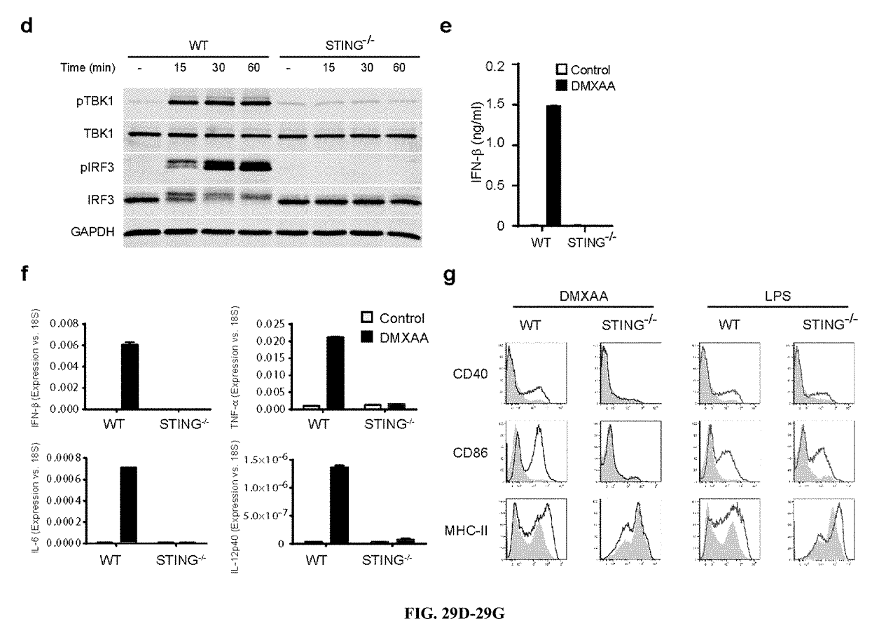

DMXAA stimulates the STING pathway in vitro. The inventors first evaluated whether DMXAA was a functional agonist of the STING pathway using mouse macrophages in vitro. STING aggregation was assessed using STING^{-/-} macrophages expressing mSTING-HA. Control macrophages presented a diffuse pattern of STING in the cytoplasm, but after one hour of incubation with DMXAA, approximately 60% of cells displayed aggregates of STING in perinuclear sites (FIG. 29a). Downstream phosphorylation of TBK1 and IRF3 was observed, which was abolished in STING^{-/-} cells (FIG. 29b) (Conlon, et al., 2013). This correlated with an increase in the apparent molecular weight of STING, which has been reported to be due to its phosphorylation (Konno, et al., 2013). STING^{-/-} macrophages reconstituted with mSTING-HA showed restored phosphorylation of TBK1 and IRF3. IFN-β secretion was detected from wild-type (WT) but not from STING^{-/-} macrophages in response to DMXAA (FIG. 29c). Similar results were observed with bone marrow-derived DCs (BM-DC) from WT versus STING^{-/-} mice (FIG. 29d-e). The inventors also used these cells to study the expression of different cytokines. IFN-β, TNF-α, IL-1β, IL-6 and IL12p35 were induced after stimulation with DMXAA in WT cells but not STING^{-/-} BM-DCs (FIG. 29f). The inventors also compared the induction of co-stimulatory molecules in BM-DCs stimulated with DMXAA or LPS. Whereas LPS induced expression of CD40, CD86 and MHC class II in both WT and STING-deficient DCs, induction with DMXAA was observed only in WT cells (FIG. 29g). Together, these data indicate that DMXAA is a strong agonist of mSTING, resulting in the production of IFN-β and other innate cytokines, and activation of DCs.

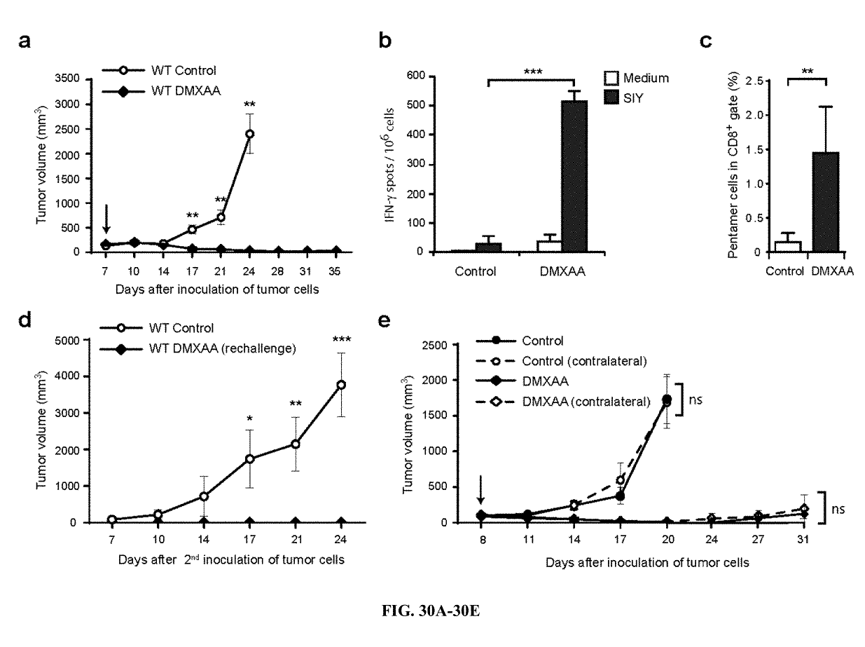

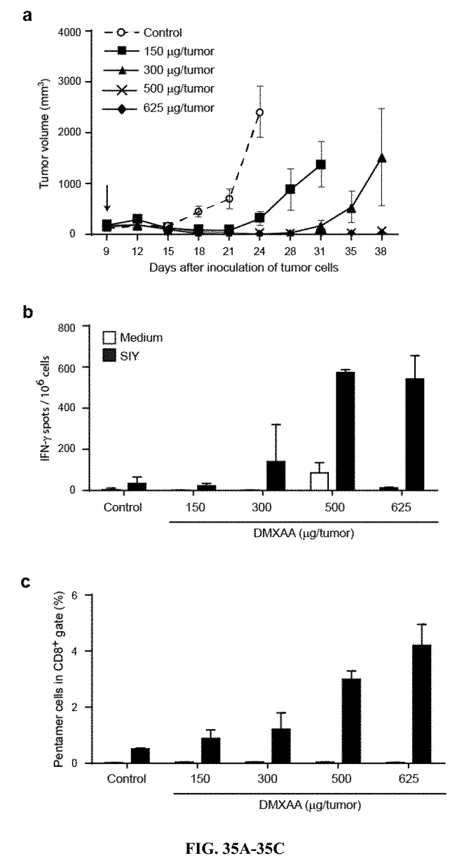

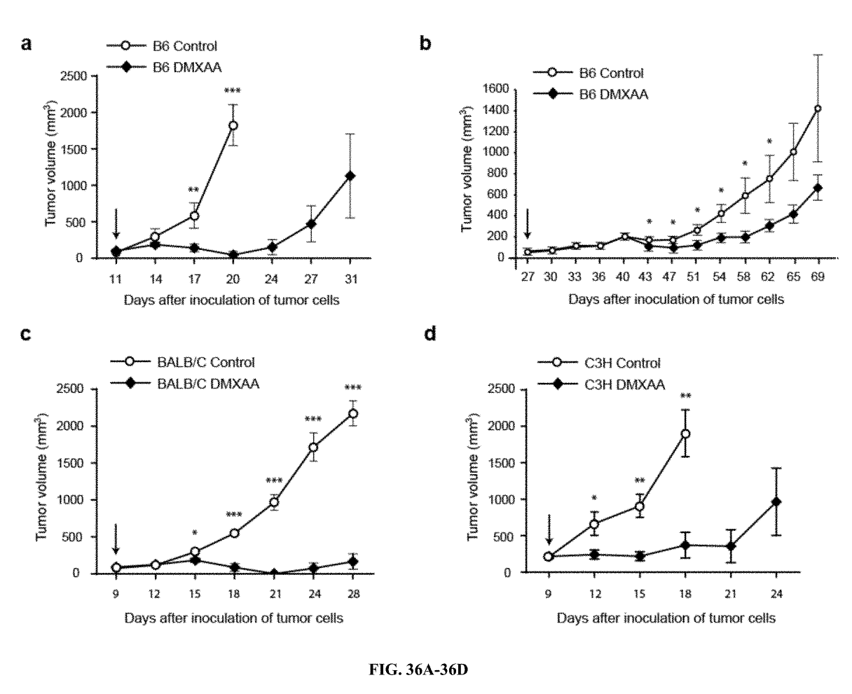

DMXAA induces strong anti-tumor immunity in vivo. In order to evaluate whether stimulation of STING could augment anti-tumor immunity in vivo, the inventors chose an intratumoral (IT) route of administration to focus activation on those APCs acquiring tumor antigens. To assess an antigen-specific immune response, the inventors utilized the B16 melanoma cell line transduced to express the model antigen SIYRYYGL (B16.SIY) (Blank, et al., 2004). B16.SIY tumor cells were inoculated into the flank of mice and injected IT with DMXAA at day 7. The dose of 500 μg of DMXAA was chosen after examining single doses ranging from 150 to 625 μg, with the highest dose of 625 μg showing unacceptable toxicity (FIG. 35). The selected dosage induced potent tumor regression in all animals and complete tumor rejection in the majority of mice (FIG. 30a). Analysis of splenocytes 5 days after treatment showed a marked increase in the frequency of SIY-specific IFN-γ-producing T cells (FIG. 30b), and high frequency of SIY-specific CD8⁺ T cells detected by SIY/K^{b} pentamer staining (FIG. 30c).

To determine whether immunologic memory was induced, mice that had rejected B16.SIY tumors were rechallenged 60 days after the initial inoculation with the same tumor cells. None of the rechallenged animals developed tumors (FIG. 30d). The inventors then investigated whether the anti-tumor immune response induced following DMXAA administration could be potent enough to reject non-injected secondary tumors. B16.SIY cells were injected in both flanks of mice but only one tumor was treated with DMXAA. Tumor regression was observed in both sites (FIG. 30e), suggesting that IT DMXAA administration can have a therapeutic effect on distant tumors. This effect was unlikely secondary to systemic distribution of the drug, since deliberate systemic administration of DMXAA via intraperitoneal (IP) administration had an inferior therapeutic effect (data not shown).

To assess whether the potent anti-tumor efficacy resulting from IT administration of DMXAA could be broadly applied, the inventors tested additional syngeneic tumor models. Treatment with DMXAA significantly reduced the growth of B16.F10 (without expression of SIY) and TRAMP-C2 tumors in C57BL/6 mice; 4T-1 tumors in BALB/c mice; and Ag104L tumors in C3H mice, indicating that the therapeutic effect of DMXAA is not restricted to a specific tumor histology or mouse genetic background (FIG. 36).

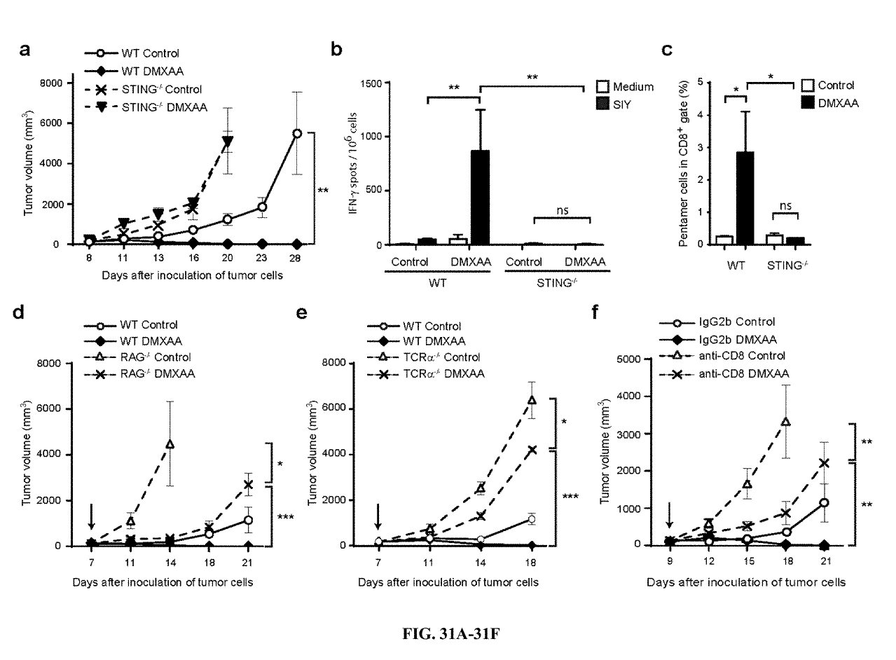

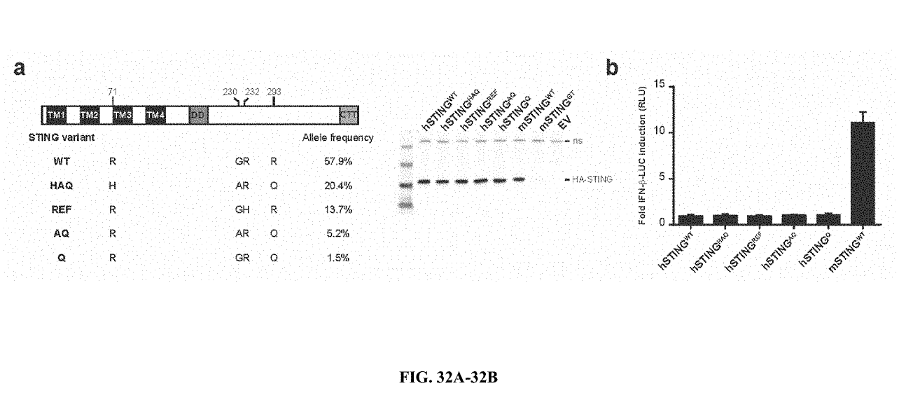

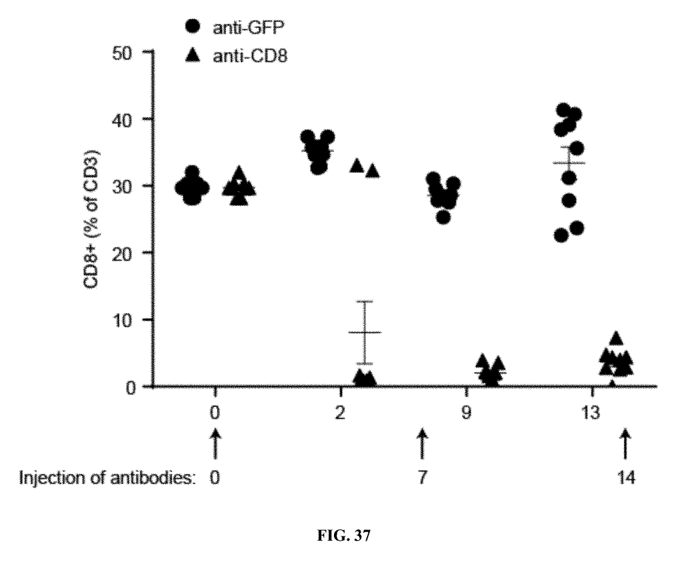

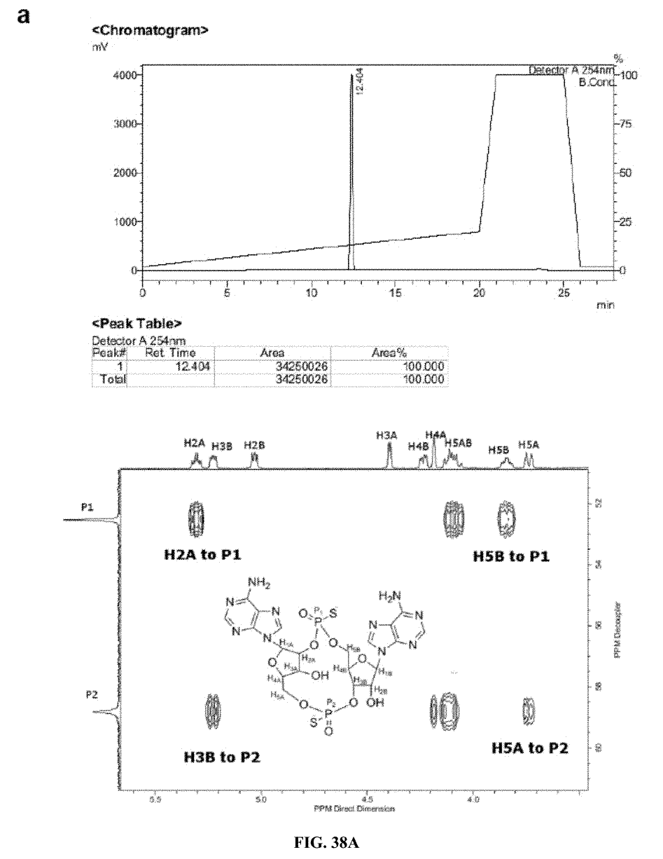

Mechanism of action of DMXAA in vivo. To test whether the therapeutic effect of DMXAA was STING-dependent, STING^{-/-} mice bearing B16.SIY tumors were used. No reduction in tumor growth was observed in response to DMXAA in the absence of host STING (FIG. 31a), and the frequencies of SIY-specific T cells were markedly reduced (FIG. 31b-c). To determine whether the adaptive immune response was required for tumor control, B16.SIY cells were inoculated into RAG2^{-/-} mice that lack mature T and B cells. DMXAA treatment lost most of its therapeutic effect in RAG2−/− hosts, although there was a partial control of tumor growth (FIG. 31d). A similar loss of therapeutic effect was observed in TCRα^{-/-} mice (FIG. 31e), and in mice depleted of CD8⁺ T cells (FIG. 31f and FIG. 37). These results indicate that a major component of the therapeutic effect of DMXAA is mediated by CD8⁺ T cells.

Identification of novel synthetic human STING-activating molecules. Having shown that the STING pathway could be harnessed to promote tumor antigen-specific CD8⁺ T cell priming leading to significant therapeutic efficacy, the inventors sought to identify compounds that could potently activate hSTING and therefore be considered for clinical translation. Cyclic dinucleotides (CDNs) have been studied as small molecule second messengers synthesized by bacteria which regulate diverse processes including motility and formation of biofilms. The immunogenicity of recombinant protein antigens can be augmented with CDNs used as an adjuvant, giving CDNs a potential application towards vaccine development. The inventors sought to develop novel synthetic CDN compounds with increased activity in human cells as well as the ability to engage all known polymorphic STING molecules. The availability of CDN-STING crystal structures, along with recent results describing hSTING allele/CDN-dependent signaling relationships, facilitated structure-based studies to design CDN compounds with increased activity. The inventors synthesized compounds that varied in purine nucleotide base, structure of the phosphate bridge linkage, and substitution of the non-bridging oxygen atoms at the phosphate bridge with sulfur atoms. Native CDN molecules are sensitive to degradation by phosphodiesterases that are present in host cells or in the systemic circulation. The inventors found that Rₚ, Rₚ (R,R) dithio-substituted diastereomer CDNs were both resistant to digestion with snake venom phosphodiesterase and induced higher expression of IFN-β in human THP-1 cells compared to the Rₚ, Rₛ (R,S) dithio-substituted diastereomers or unmodified CDNs.

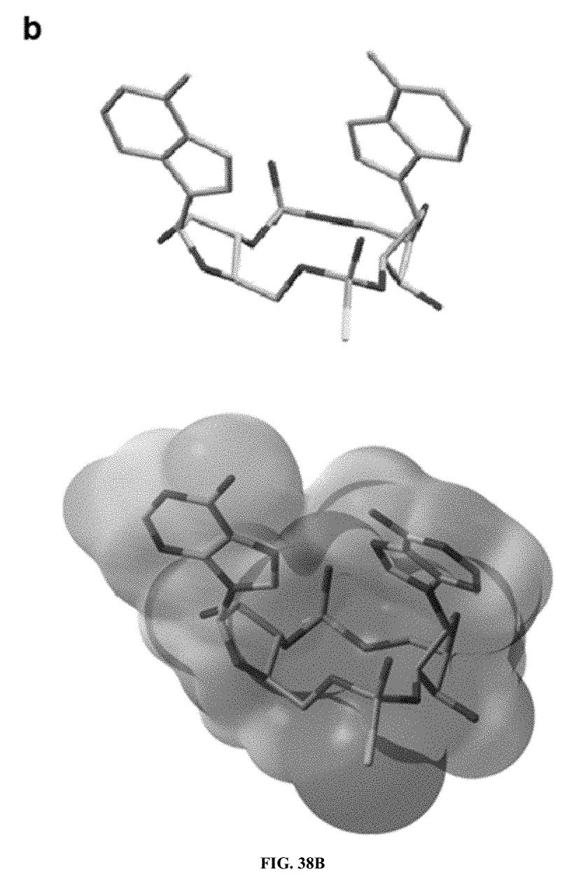

To increase their affinity for STING, CDNs were also synthesized with a phosphate bridge configuration containing both 2′-5′ and 3′-5′ linkages, termed “mixed linkage” (ML), as found in endogenous human CDNs produced by cGAS. The synthesis of dithio mixed-linkage CDNs via modifications of literature procedures, resulted in both R,R- and R,S dithio diastereomers which were purified and separated by a combination of silica gel and C18 reverse phase prep-HPLC chromatography, affording CDNs with ≧95% purity as shown for ML RR-S2 CDA in FIG. 38A, upper panel. The spectra for both ¹H NMR (data not shown) and the ³¹P NMR (y-axis of FIG. 38A, lower panel) were consistent with ML RR-S2 CDA. Direct evidence for the regiochemistry of the phosphodiester linkages was obtained by ¹H-¹H COSY (correlation spectroscopy for assignment of ribose protons shown on x-axis of FIG. 38A (lower panel), in combination with a ¹H-³¹P HMBC (heteronuclear multiple-bond correlation spectroscopy) two-dimensional NMR (FIG. 38A, lower panel). The three-dimensional X-ray crystal structure of ML RR-S2 CDA confirms the presence of the 2′-5′, 3′-5′mixed phosphodiester linkage and a dithio [Rₚ, Rₚ] diastereomer configuration (FIG. 38B).

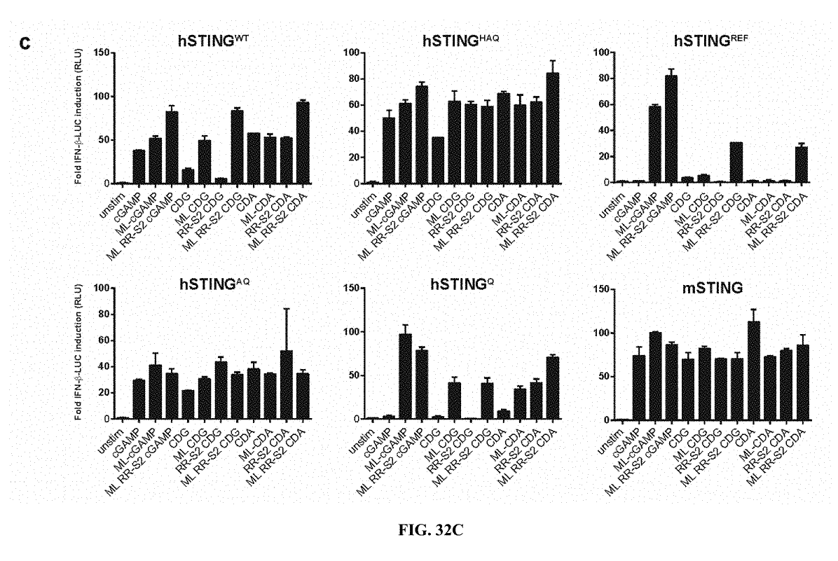

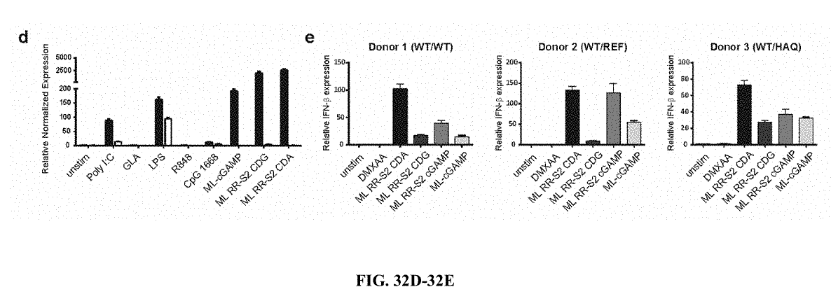

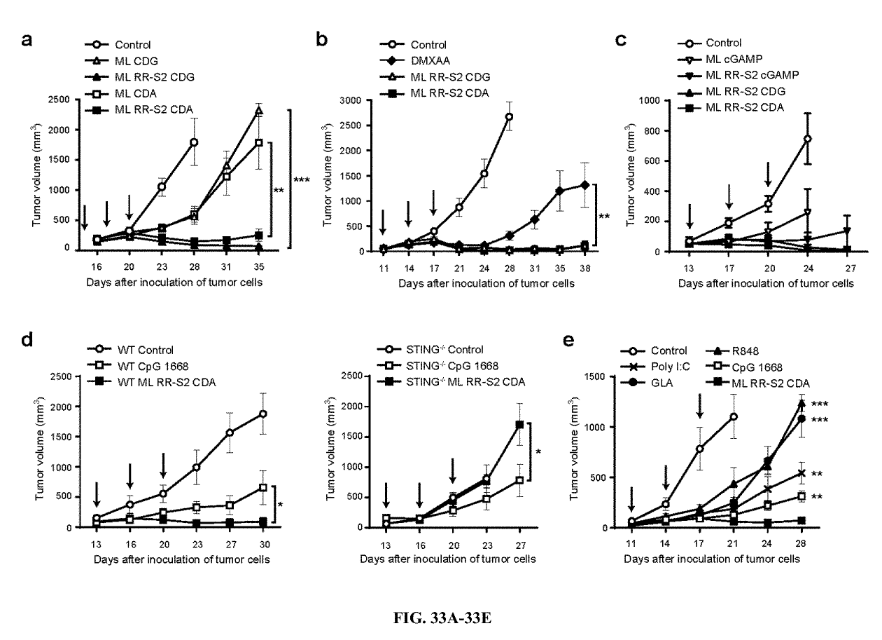

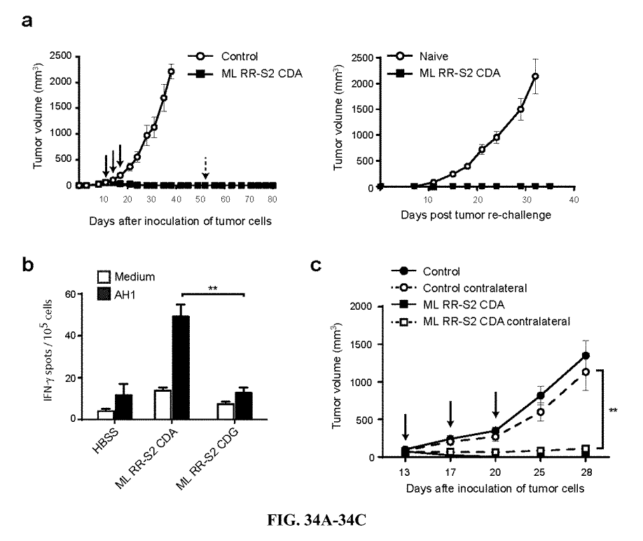

Novel synthetic CDNs activate all known human STING alleles. Single nucleotide polymorphisms in the hSTING gene have been shown to affect the responsiveness to bacterial-derived canonical CDNs (Diner, et al., 2013; Gao, et al., 2013). Five haplotypes of hSTING have been identified (WT, REF, HAQ, AQ and Q alleles), which vary at amino acid positions 71, 230, 232 and 293. (FIG. 32A, left) (Jin, et al., 2011; Yi, et al., 2013). To test the responsiveness of the five hSTING variants to synthetic CDNs, the inventors created stable HEK293T cell lines (deficient in endogenous STING) expressing each of the full length hSTING variants. Similar levels of STING protein were expressed in each of the cell lines (FIG. 32A, right). As expected, DMXAA potently activated mSTING, but failed to activate any of the five hSTING alleles (FIG. 32B). Cells expressing hSTING^{REF} responded poorly to stimulation with the bacterial CDN compounds cGAMP, CDA, and CDG, but were responsive to the endogenously produced cGAS product, ML-cGAMP (Diner, et al., 2013) (FIG. 32C). Interestingly, the hSTING^{Q} allele was also refractory to the bacterial CDNs. Cells expressing mSTING were responsive to all of the CDNs tested. Cells transformed with either an empty vector or expressing a non-functional mutant (I199N) STING protein (Goldenticket) (Sauer, et al., 2011) were not responsive to any of the compounds (data not shown). In contrast, the natural ligand ML-cGAMP as well as the dithio, mixed-linkage CDN derivatives (ML RR-CDA, ML RR-S2 CDG, and ML RR-S2 cGAMP) potently activated all five hSTING alleles, including the refractory hSTING^{REF} and hSTING^{Q} alleles (FIG. 32C).

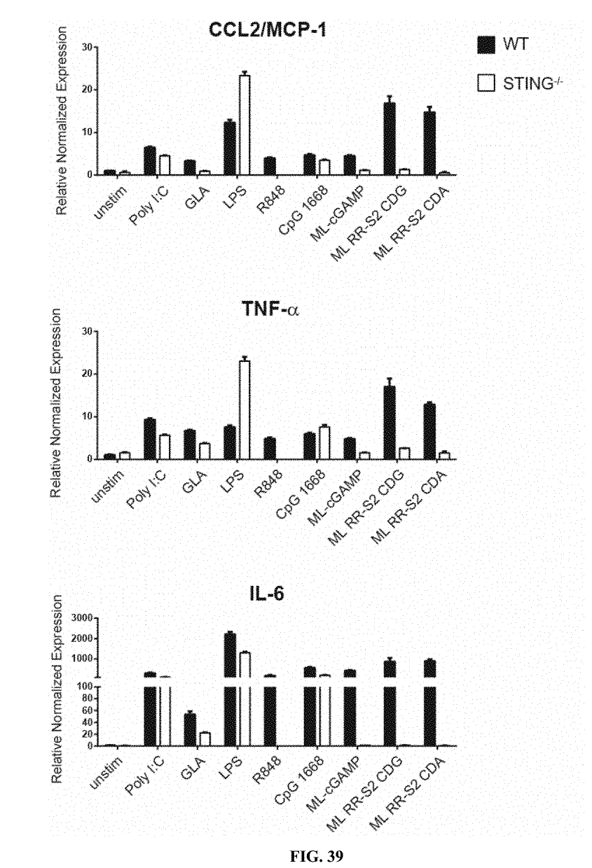

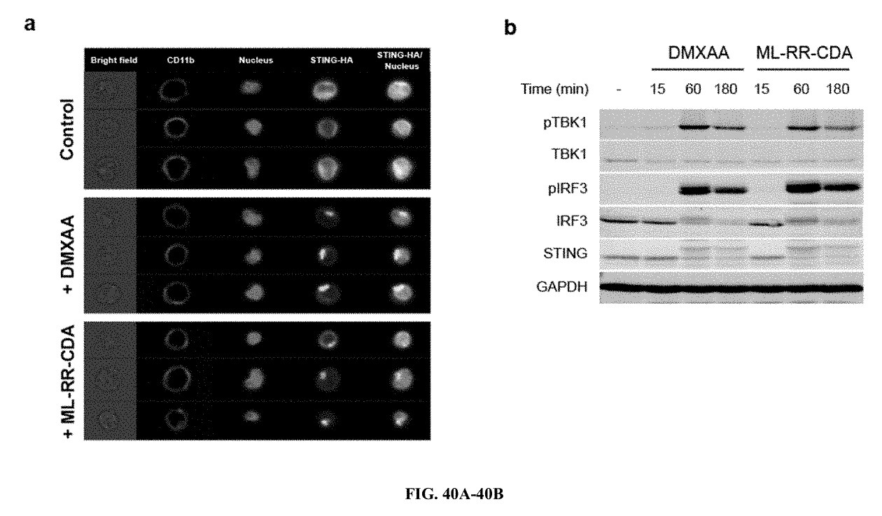

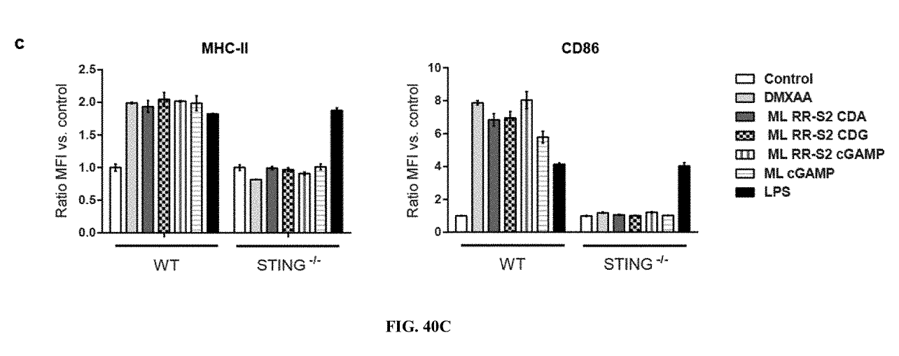

CDN derivatives potently induce STING-dependent signaling in murine and human immune cells. To determine whether CDNs activated downstream STING signaling, the inventors assessed murine bone marrow macrophages (BMMs) isolated from WT C57BL/6 and STING^{-/-} (Goldenticket) mice for induction of IFN-β and other cytokines. Synthetic dithio mixed-linkage CDNs (ML RR-S2 CDA and ML RR-S2 CDG) induced the highest expression of pro-inflammatory cytokines on a molar equivalent basis, as compared to endogenous ML-cGAMP and the TLR3 and TLR4 agonists poly I:C and LPS (respectively) (FIG. 32D). The modified CDNs did not induce signaling in STING^{-/-} BMMs, whereas, as expected, TLR agonists were still active. Similar results were seen when induction of TNF-α, IL-6, and MCP-1 were measured (FIG. 39). To examine activation of STING signaling in primary human cells, the inventors stimulated PBMCs from a panel of human donors harboring different STING alleles and measured induction of IFN-β. In contrast to DMXAA, dithio-modified mixed linkage CDNs induced IFN-β expression across multiple human donors (FIG. 32E). ML RR-S2 CDA was also found to induce aggregation of STING in mouse BMM, and induce phosphorylation of TBK1 and IRF3 (FIG. 40A-40B). All of the modified CDNs tested also enhanced MHC class I and expression of co-stimulatory markers in a STING-dependent manner (FIG. 40C). Thus, ML RR-S2 CDNs are viable clinical candidates capable of activating the human STING pathway.

Intratumoral delivery of synthetic CDN derivatives results in profound anti-tumor efficacy in established B16 melanoma. To evaluate whether modified dithio ML CDN compounds also had anti-tumor activity, mice bearing established B16.F10 tumors (without SIY expression) were treated with three IT injections of CDN derivatives over a one-week period. While treatment with ML c-di-AMP (ML-CDA) and ML c-di-GMP (ML-CDG) had modest effects on tumor growth, the R,R dithio derivatives profoundly inhibited tumor growth (FIG. 33A), and were significantly more potent than DMXAA (FIG. 33B), and endogenous ML-cGAMP (FIG. 33C). However, ML RR-S2 CDG was reactogenic, and some mice developed open wounds in the treated tumor that did not heal. Lower dose levels of ML RR-52 CDG were not efficacious, indicating that this molecule had a narrow therapeutic index. In contrast, no injection site reactogenicity was observed with ML RR-52 CDA, and several mice developed vitiligo upon fur regrowth following complete eradication of the treated tumor (data not shown).

To determine whether the CDN-induced anti-tumor efficacy was STING-dependent, the inventors compared activity in B16 tumor-bearing WT (C57BL/6) and STING^{-/-} mice. CDN therapeutic efficacy was completely lost in STING^{-/-} mice (FIG. 33D). ML RR-S2 CDA demonstrated significantly increased potency as compared to CpG-based TLR9 agonists (Kawarada, et al., 2001) in B16 tumor-bearing mice, and also compared to multiple other TLR agonists given IT at the same doses (FIG. 33E).

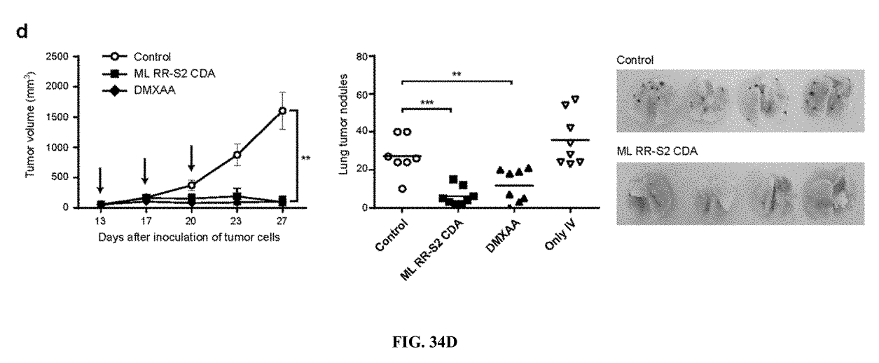

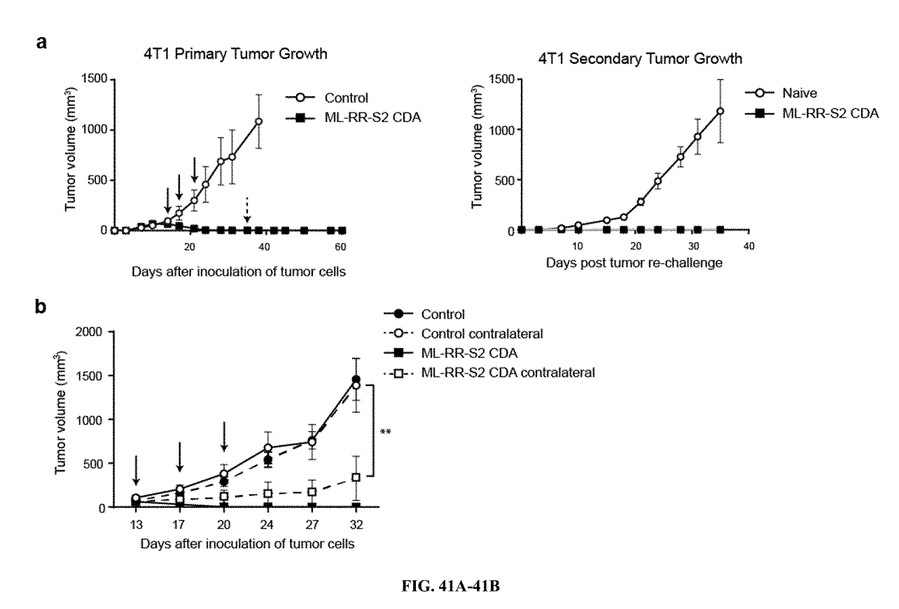

ML RR-S2 CDA induces lasting immune-mediated tumor rejection in multiple tumor types. To test different genetic backgrounds, BALB/c mice bearing established CT26 colon or 4T1 mammary carcinomas were treated ML RR-S2 CDA. All treated animals showed significant and durable tumor regression. Mice that were cured of their primary tumor were completely resistant to re-challenge in both tumor models (FIG. 34A and FIG. 41A), and improved immune responses were observed against the endogenous CT26 rejection antigen AH1 (Slansky, et al., 2000) (FIG. 34B). IT injection of ML RR-S2 CDA into one tumor in BALB/c mice bearing bilateral CT26 or 4T1 tumors also demonstrated significant regression of the contralateral untreated tumor (FIG. 34C and FIG. 41B). The inventors also implanted B16 melanoma in C57BL/6 mice, and seven days later gave intravenously infused B16 melanoma cells. The two-week old established flank tumors were treated with ML RR-S2 CDA, DMXAA or HBSS control, and three weeks later lung metastases were enumerated. Mice treated in the flank tumor with ML RR-S2 CDA showed more significant inhibition of growth of distant lung metastases than DMXAA (FIG. 34D). Together, these results demonstrate that IT injection with ML RR-S2 CDA eradicates multiple tumor types and primes an effective systemic CD8⁺ T cell immune response that significantly inhibits the growth of distal untreated lesions.

### Methods

Cells and Cell Isolations. The cells used for the in vivo experiments were: the C57BL/6-derived melanoma cell lines B16.F10 and B16.F10.SIY (henceforth referred to as B16.SIY), the breast cancer OT-1 and 4T1 cell lines, the prostate cancer TRAMP-C2 cell line, the colon cancer CT26 cell lines, all originally purchased from ATCC. The fibrosarcoma Ag104L cell line was gifted by Dr. Hans Schreiber, University of Chicago. All cells were maintained at 37° C. with 7.5% CO₂ in DMEM supplemented with 10% heat-inactivated FCS, penicillin, streptomycin, L-arginine, L-glutamine, folic acid, and L-asparagine.

Immortalized WT and STING^{-/-} macrophages were obtained as described in Roberson et al. (Roberson, et al., 1988). The WT macrophages were obtained from Dr. K Fitzgerald (U. Massachusetts). Non-immortalized macrophages were derived from the bone marrow of WT (C57BL/6) or STING^{-/-} mice and cultured in BMM media (RPMI media with 5% CSF, 5% FBS, 1× L-glutamine, 1× Pen/Strep) for 7 days prior to use. Bone marrow-derived dendritic cells (BMDCs) from WT and STING^{-/-} mice were generated by culturing cells from the tibiae and femurs in the presence of rmGM-CSF (20 ng/ml; BioLegend) for 9 days. After the incubation, the phenotype of cells with specific antibodies confirmed that >90% of the cells were CD11c⁺, CD11b⁺ or CD11b⁻, and CD8⁻,Cd4⁻ and CD19⁻. Human PBMCs were isolated by density-gradient centrifugation using Ficoll-Paque Plus (GE Healthcare).

For stable overexpression of HA-STING in STING^{-/-} macrophages, sequence encoding full-length mSTING were amplified from pUNOI-mSTING plasmid (Invivogen) and cloned into the empty pMX-IRES-GFP vector. Stable HEK 293T STING-expressing cell lines were generated with MSCV2.2 retroviral plasmids which contain STING cDNA cloned upstream of an IRES in frame with GFP. hSTING(REF)-HA, hSTING(WT)-HA, hSTING(HAQ)-HA, hSTING(Q)-HA and mSTING(WT)-HA retroviral plasmids were obtained from the Vance Laboratory at UC Berkeley. hSTING(AQ)-HA was derived from hSTING(Q)-HA using a QuickChange Site-Directed Mutagenesis kit (Stratagene). Retroviral vectors were transfected into the amphotropic Phoenix packaging cell line using Lipofectamine (Invitrogen). After two days viral supernatants were harvested and used for transduction of STING^{-/-} macrophages or HEK 297 cells. GFP⁺ cells were sorted in ACSAria (BD) or MoFlow cell sorters.

ImageStream analysis of STING aggregates. STING^{-/-} macrophages overexpressing STING-HA tag were stimulated for 1 hour with 50 μg/m1 of DMXAA resuspended in 7.5% of NaHCO₃, 50 μM of ML RR-S2 CDA resuspended in HBSS, or only the vehicles as control. After the incubation, cells were stained with anti-CD11b-APC (M1/70; BioLegend), rabbit anti-HA-tag (C29F4; Cell Signaling) and anti-Rabbit IgG-PE (Invitrogen), and DAPI (Invitrogen). Single cell images were acquired in the ImageStreamxMark II (Amnis) and data were analyzed using IDEAS software.

Western blot analysis. WT, STING^{-/-} macrophages, and STING^{-/-} macrophages overexpressing STING-HA or an empty vector were stimulated with 50 μg/ml DMXAA for 0, 15, 60 or 180 minutes; BM-DCs from WT or STING^{-/-} mice were stimulated with 25 μg/ml

DMXAA for the same time-points. Proteins were extracted with Triton-X buffer (150 mM sodium chloride, 50 mM Tris, 1% Triton-X, pH 8.0) with proteinase inhibitors (Thermo scientific) and phosphatase inhibitors (Sigma). 30 μg of protein was electrophoresed in 10% SDS-PAGE gels and transferred onto Immobilon-FL membranes (Millipore). Blots were incubated with antibodies specific for phosphorylated TBK1 (Ser172), phosphorylated IRF3 (Ser396), total TBK1, STING and GAPDH (Cell Signaling) or total IRF3 (Invitrogen). Proteins from HEK 293T lines stably expressing STING were extracted with M-PER (Thermo Scientific). 6 μg of protein was loaded onto a 4-12% MES NuPAGE gel (Life Technologies), transferred to nitrocellulose, and probed with anti-HA antibody (Santa Cruz). Anti-rabbit IRDye 680RD label secondary antibody was used for visualization of bands with the Odyssey Imaging system (LI-COR).

Murine IFN-β ELISA. WT or STING^{-/-} macrophages and BM-DCs from WT or STING^{-/-} mice were stimulated with 50 μg/ml DMXAA. Conditioned media were collected after 4 hours. IFN-β concentration was assessed using VeriKine™ Mouse Interferon Beta ELISA Kit (PBL interferon source).

Quantitative RT-PCR analysis of cytokines. BM-DCs from WT or STING^{-/-} mice were stimulated with 25 μg/ml DMXAA or 100 ng/ml LPS for 4 hours. Total RNA was isolated using the RNeasy® kit (Qiagen) and incubated with Deoxyribonuclease I, Amplification Grade (Invitrogen). cDNA was synthesized using High Capacity cDNA Reverse Transcription Kit (Applied Biosystem) and expression of cytokines was measured by real-time qRT-PCR using specific primers/probes for mouse INF-β, TNF-α, IL-6 and IL-12p40, using a 7300 Real Time PCR system (Applied Biosystem). The results are expressed as 2^{-ΔCt} using 18s as endogenous control.

WT BMM were stimulated with CDN at 5 μM in HBSS with the addition of Effectene (Qiagen) transfection reagent (per kit protocol). Human PBMCs were stimulated in normal RPMI media using with 10 μM of each CDN or 100 μg/ml DMXAA. After a 6 hr incubation, cells were harvested and assessed by real-time qRT-PCR for gene expression of IFN-β1, MCP-1, TNF-α) and IL-6 using the PrimePCR RNA purification and cDNA analysis system, and run on the CFX96 gene cycler (BioRad). Relative normalized expression was determined by comparing induced target gene expression to unstimulated controls, using the reference genes Gapdh and Ywhaz, genes confirmed to have a coefficient variable (CV) below 0.5 and M value below 1, and thus did not vary with different treatment conditions.

Expression of activation markers by flow cytometry. BM-DCs from WT or STING^{-/-} mice were stimulated with 25 μg/ml DMXAA or 100 ng/ml LPS for 12 hours, or with 50 μM of each CDN for 24 hours. After stimulation, cells were pre-incubated for 15 min with anti-CD16/32 monoclonal antibody (93) to block potential nonspecific binding and then with specific antibodies: anti-CD11c-Pe-Cy7 or APC (N418), anti-CD11b-PerCP-Cy5.5 (M1/70), anti-CD40-PE (3/23), anti-CD80-APC (16-10A1), anti-CD86-FITC or PE (GL1) and anti-IA/IE-PB or FITC (M5/114.15.2). Stained cells were analyzed using LSR II cytometer with FACSDiva software (BD) or FACSVerse with FACSuite software. Data analysis was conducted with FlowJo software (Tree Star).

Mice. C57BL/6, BALB/c, C3H/He and TCRα^{-/-} mice were obtained from Jackson and Charles River. RAG2^{-/-} mice were obtained from Taconic. Tmem173^{-/-} (STING-deficient) mice were provided by Dr. G. Barber (University of Miami), and STING^{-/-} (goldenticket) mice were purchased from Jackson.

In vivo tumor experiments. 10⁶ of B16-SIY tumor cells, 5×10⁴ B16.F10 tumor cells, 10⁵ 4T1 and CT26, or 10⁶ other tumor cells were injected s.c. in 100 μl DPBS or HBSS on the right flank of mice. Following tumor implantation, mice were randomized into treatment groups. When tumors were 100-200 mm³ in volume (5-7 mm wide), either one single or three doses of DMXAA resuspended in 7.5% of NaHCO₃, or CDNs formulated in HBSS or vehicle control, were injected IT. Measurements of tumors were performed twice per week using calipers, and the tumor volume was calculated with the formula: V=(length×width²)/2. In some experiments, tumor-free survivors were rechallenged with tumor cells on the opposite flank several weeks after the injection of the primary tumor. Naïve mice were used as controls. For the contralateral experiments, mice were implanted on both flanks and only one tumor was treated. For the B16 melanoma lung metastasis experiments, mice were implanted on the flank with 5×10⁴ cells B16.F10 on day 0, and then injected intravenously with 1×10⁵ cells on day 7. Lungs were harvested on day 28. Administration of compounds, measurements of tumors and counting of lung tumors were performed in a blinded fashion.

CD8⁺ T cell depletion. For depletion of CD8⁺ T cells, mice were injected IP weekly with rat mAb to mouse CD8 (43.2) or isotype control IgG2b (BioXcell) at a dose of 250 μg per mouse. This regimen of administration resulted in approximately 99% depletion of CD8⁺ T cells from the peripheral blood, as evaluated by flow cytometry using a different clone for anti-CD8 (53-6.7; Biolegend).

IFN-γ ELISPOT and SIY-pentamer staining. Splenocytes were analyzed 5 days after the first IT injection of DMXAA. For the ELISPOTs, 10⁶ splenocytes were plated per well and stimulated overnight with SIY peptide (160 nM) or AH1 (1 μM) peptide, with PMA (50 ng/ml) plus ionomycin (0.5 μM) as a positive control, or medium as negative control. Spots were developed using the BD mouse IFN-γ kit according to the manufacturer's instructions and the number of spots was measured using an Immunospot Series 3 Analyzer and analyzed using ImmunoSpot software (Cellular Technology Ltd). For SIY-pentamer staining, splenocytes were preincubated for 15 min with anti-CD16/32 monoclonal antibody (93) to block potential nonspecific binding, and labeled with PE-MHC class I pentamer (Proimmune) consisting of murine H-2K^{b} complexed to SIYRYYGL (SIY) peptide, anti-TCRβ-AF700 (H57-597), anti-CD8-Pacific Blue (53-6.7), anti-CD4-Pacific Orange (RM4-5) (all antibodies from BioLegend) and the Fixable Viability Dye eFluor 450 (eBioscience). Stained cells were analyzed using LSR II cytometer with FACSDiva software (BD). Data analysis was conducted with FlowJo software (Tree Star).

Preparation of natural cyclic dinucleotide STING ligands and synthetic derivative molecules. Modified CDN derivative molecules were synthesized according to modifications of the “one-pot” Gaffney procedure, described previously (Gaffney, et al., 2010). Synthesis of CDN molecules utilized phosphoramidite linear coupling and H-phosphonate cyclization reactions. Synthesis of dithio CDNs was accomplished by sulfurization reactions to replace the non-bridging oxygen atoms in the internucleotide phosphate bridge with sulfur atoms. For example, synthesis of dithio-(Rp,Rp)-[cyclic[A(2′,5′)pA(3′,5′)p]], shown as ML RR-S2 CDA in FIG. 38B, on a five millimole scale was achieved with 5′-O-DMTr-3′-O-TBDMS-Adenosine (N-Bz)-2′-CEP and the H-phosphonate derived from 5′-O-DMTr-3′-O-TBDMS-Adenosine (N-Bz)-3′-CEP. The phosphorus III intermediates generated upon formation of the linear dimer (phosphite triester stage) and cyclic dincucleotide (H-phosphonate diester stage) were sulfurized by treatment with 3-((N,N-dimethylaminomethylidene)amino)-3H-1,2,4-dithiazole-5-thione (DDTT) and 3-H-1,2-benzodithiol-3-one, respectively. The crude reaction mixture obtained after the second sulfurization was chromatographed on silica gel to generate a mixture of the RR- and RS-diastereomers of fully protected ML S2 CDA. Benzoyl and cyanoethyl deprotection using methanol and concentrated aqueous ammonia generated bis-TBS-ML-S2 CDA as a mixture of RR- and RS-diastereomers which were separated by C-18 prep HPLC. The purified bis-TBS-ML RR-S2 CDA was deprotected with TEA-3HF, neutralized with 1 M triethylammonium bicarbonate and desalted on a C18 SepPak to give ML RR-S2 CDA as the bis-triethylammonium salt in >95% purity. Alternatively, the TEA groups were exchanged with either sodium or ammonium counter ions by ion exchange, lyophilized, and resuspended in 10 mM Tris pH7/1 mM EDTA buffer to ˜5 mg/mL, and filter sterilized through a 0.2 micron filter, resulting in a final product that was ≧95% purity as determined by analytical HPLC (FIG. 38A). High resolution Fourier transform ion cyclotron resonance mass spectroscopy (FT-ICR) confirmed the expected elemental formula: [M-H]⁻ calculated for C₂₀H₂₃N₁₀O₁₀P₂S₂ 689.0521; found 689.0514. The spectra for both ¹H NMR (data not shown) and the ³¹P NMR (y-axis of FIG. 38A) were consistent with ML RR-S2 CDA. Direct evidence for the regiochemistry of the phosphodiester linkages was obtained by ¹H-¹H COSY (correlation NMR spectroscopy) for assignment of ribose protons (shown on x-axis of FIG. 38A) in combination with a ¹H-³¹P HMBC (heteronuclear multiple-bond correlation spectroscopy) experiment. Prior to use in experiments, all synthetic CDN preparations were verified by LAL assay to be endotoxin free (<1 EU/mg).

Human STING sequencing. Genomic DNA was isolated from 10⁴ PBMCs using Quick Extract DNA Extraction Solution (Epicentre) and used to amplify regions of exon 3, 6, and 7 of hSTING. Primers for amplification and sequencing are listed in Table 1.

TABLE 1
List of primers used in real time PCR and for sequencing STING alleles.

| | | | |
|---|---|---|---|
| Gene | Forward | Reverse | Probe |

Cytokines

| | | | |
|---|---|---|---|
| IFN-β | GGAAAGATTGACGTGGGAGA | CCTTTGCACCCTCCAGTAAT | CTGCTCTC |
| | (SEQ IDN O: 14) | (SEQ ID NO: 21) | (SEQ ID NO: |
| | | | 28) |
| TNF-α | CTGTAGCCCACGTCGTAGC | GGTTGTCTTTGAGATCCATGC | CCAGGAGG |
| | (SEQ ID NO: 15) | (SEQ ID NO: 22) | (SEQ ID NO: |
| | | | 29) |
| IL-6 | GCTACCAAACTGGATATAATCAGGA | CCAGGTAGCTATGGTACTCCAGAA | TTCCTCTG |
| | (SEQ ID NO: 16) | (SEQ ID NO: 23) | (SEQ ID NO: |
| | | | 30) |
| IL-12p40 | CCTGCATCTAGAGGCTGTCC | CAAACCAGGAGATGGTTAGCTT | GACTCCAG |
| | (SEQ ID NO: 17) | (SEQ ID NO: 24) | (SEQ ID NO: |
| | | | 31) |

STING alleles

| | | |
|---|---|---|
| hSTING | GCTGAGACAGGAGCTTTGG | AGCCAGAGAGGTTCAAGGA |
| exon 3 | (SEQ ID NO: 18) | (SEQ ID NO: 25) |
| hSTING | GGCCAATGACCTGGGTCTCA | CACCCAGAATAGCATCCAGC |
| exon 6 | (SEQ ID NO: 19) | (SEQ ID NO: 26) |
| STING- | TCAGAGTTGGGTATCAGAGGC | ATCTGGTGTGCTGGGAAGAGG |
| HAQ | (SEQ ID NO: 20) | (SEQ ID NO: 27) |

Luciferase Assay. 10⁴ HEK 293T cells were seeded in 96-well plates and transiently transfected (Lipofectamine 2000) with human IFN-β firefly reporter plasmid⁴⁶ and TK-Renilla luciferase reporter for normalization. The following day, cells were stimulated with 10 μM of each CDN or 100 μg/ml DMXAA using digitonin permeabilization (50 mM HEPES, 100 mM KCL, 3 mM MgC12, 0.1 mM DTT, 85 mM Sucrose, 0.2% BSA, 1 mM ATP, 0.1 mM GTP, 10 μg/ml digitonin) to ensure uniform uptake. After 20 min, stimulation mixtures were removed and normal media was added. After a total of 6 hours, cell lysates were prepared and reporter gene activity measured using the Dual Luciferase Assay System (Promega) on a Spectramax M3 luminometer.

ML RR-S2 CDA Crystal Structure and Electrostatic Potential Surface. The X ray structure was determined at UC Berkeley College of Chemistry X-ray Crystallography Facility (Antonio DiPasquale, PhD). X-ray quality crystals were grown from a saturated wet ethanol solution followed by the slow vapor diffusion of acetone, which was then followed by the slow vapor diffusion of hexane to deposit the crystalline material. A colorless plate 0.050×0.040×0.010 mm in size was mounted on a Cryoloop with Paratone oil. Data were collected in a nitrogen gas stream at 100(2) K using phi and omega scans. Crystal-to-detector distance was 60 mm and exposure time was 10 seconds per frame using a scan width of 1.0°. Data collection was 100.0% complete to 67.000° in θ. A total of 113285 reflections were collected covering the indices, −19<=h<=19, −24<=k<=24, −26<=l<=29. 14929 reflections were found to be symmetry independent, with an Rᵢₙₜ of 0.0445. Indexing and unit cell refinement indicated a primitive, orthorhombic lattice. The space group was found to be P 21 21 21 (No. 19). The data were integrated using the Bruker SAINT software program and scaled using the SADABS software program. Solution by iterative methods (SHELXT) produced a complete heavy-atom phasing model consistent with the proposed structure. All non-hydrogen atoms were refined anisotropically by full-matrix least-squares (SHELXL-2014). All hydrogen atoms were placed using a riding model. Their positions were constrained relative to their parent atom using the appropriate HFIX command in SHELXL-2014. Absolute stereochemistry was unambiguously determined to be R at all chiral centers. Gaussian 09 (Revision A.02) was used to optimize the structure of the dianion monomer using the B3LYP/6-31G(d) level of theory starting from the coordinates determined from the X-ray diffraction experiment. Once a stationary point in the optimization was found, an electrostatic potential surface was calculated for the optimized structure.

Statistical analysis. Student's paired t-test was used to calculate two-tailed p values to estimate statistical significance of differences between two treatment groups using Prism 6 software. Statistically significant P values are labeled in the figures and the legends with asterisks.

### ************************

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.

Abe, et al., Molecular cell. 50(1):5-15, 2013.

Ablasser, etal. Nature, 498: 380-384, 2013.

Ahn, et al., Proc Natl Acad Sci USA. 109(47): 19386-19391, 2012.

Apetoh, et al., Nat Med 13(9): 1050-1059, 2007.

Blank, et al., Cancer Res. 64: 1140-1145, 2004.

Burdette, et al. Nature. 478(7370): 515-518, 2011.

Conlon, et al. J Immunology, 2013.

Diamond, et al. J Exp Med, 208(10): 1989-2003, 2011.

Diner, et al. Cell reports 3: 1355-1361, 2013.

Dubesnky, et al., Therapeutic Advances in Vaccines., 2013.

Ebensen, et al., Vaccine, 29: 5210-5220, 2011.

Ebensen, et al., Vaccine 25: 1464-1469, 2007.

Ebensen, et al., CVI 14: 952-958, 2007.

Fuertes, et al., J Exp Med. 208(10): 2005-2016, 2011.

Fuertes, et al., Trends in immunology. 34(2): 67-73, 2013.

Gaffney, et al., Organic letters 12: 3269-3271, 2010.

Gajewski, Clin Cancer Res. 12(7 Pt 2): 2326s-2330s, 2006.

Gajewski, et al. Cancer J. 16(4): 399-403, 2010.

Gajewski, et al., Curr Opin Immunol. 25(2): 268-276, 2013.

Galon, et al., Science. 313(5795): 1960-1964, 2006.

Gao, et al. Cell, 1-14, 2013.

Ghiringhelli, et al. Nat Med. 15(10): 1170-1178, 2009.

Gray, et al. Cellular immunology 278: 113-119, 2012.

Hamid, et al. J Transl Med. 9: 204, 2011.

Harlin, et al. Cancer Res. 69(7): 3077-3085, 2009.

Henry, et al., J Exp Med. 204(5): 987-994, 2007.

Hoebe, et al. Nat Immunol. 4(12): 1223-1229, 2003.

Hwang, et al., Gynecol Oncol. 124(2):192-8, 2011.

Ishii, et al., Nat Immunol, 7(1): 40-48, 2006.

Ishikawa, et al., Nature. 461(7265): 788-792, 2009.

Jin, et al., Genes and immunity, 12: 263-269, 2011.

Jounai, et al., Frontiers in cellular and infection microbiology. 2:168, 2012.

Kawarada, et al. J Immunol. 167: 5247-5253, 2001.

Konno, et al., Cell. 155, 688-698, 2013.

Kono & Rock, Nat Rev Immunol. 8(4): 279-289, 2008.

Kroemer, et al., Annu Rev Immunol. 31: 51-72, 2013.

Lande, et al., Nature. 449(7162): 564-569, 2007.

Li, et al., Cancer Res. 68(17): 6889-6895, 2008.

Mahmoud, et al., J Clin Oncol. 29(15): 1949-1955, 2011.

Marichal, et al. Nat Med. 17(8):996-1002, 2011.

McKee, et al. Proc Natl Acad Sci USA. 110(12): E1122-1131, 2013.

Molinero, et al., American journal of transplantation. 8(1): 21-31, 2008.

Obeid, et al. Nat Med. 13(1): 54-61, 2007.

Oka, et al., Nature. 485(7397): 251-255, 2012.

Pages, et al. J Clin Oncol. 27(35): 5944-5951, 2009.

Roberson, et al., Cellular immunology, 116: 341-351, 1988.

Romling, et al., MMBR 77: 1-52, 2013.

Sancho, et al. Nature. 458(7240): 899-903, 2009.

Sauer, et al. Infection and immunity, 79: 688-694, 2011.

Sharma, et al., Immunity. 35(2): 194-207, 2011.

Slansky, et al. Immunity 13: 529-538, 2000.

Spranger, et al., Science Translational Medicine. 5(200):200ra116, 2013.

Stetson & Medzhitov, Immunity. 24(1): 93-103, 2006.

Sun, et al., Science. 339(6121): 786-791, 2013.

Takaoka, et al., Nature. 448(7152): 501-505, 2007.

Twiny, et al., Clin Cancer Res. 17(20): 6467-6481, 2011.

Unterholzner, et al., Nat Immunol 11(11): 997-1004, 2010.

Wolchok, et al. N Engl J Med 369: 122-133, 2013.

Wu, et al., Science. 339(6121): 826-830, 2013.

Yan, et al., Bioorg. & Med. Chem Lett. 18: 5631-5634, 2008.

Yanai, et al., Nature 462(7269): 99-103, 2009.

Yi, et al. PloS one 8, e77846, 2013.

Zhou, et al., Eur J Immunol. 35(3): 822-830, 2005.

Zhou, et al., J Viral. 83(4): 1625-1634, 2009.

1. A method of treating cancer in a subject comprising administering to the subject an effective amount of a stimulator of interferon genes (STING) agonist, wherein the STING agonist is administered intratumorally.

2. The method of claim 1, wherein the STING agonist is a nucleic acid, a protein, a peptide, or a small molecule.

3. The method of claim 2, wherein the STING agonist is a small molecule.

4. The method of claim 3, wherein the small molecule is a cyclic dinucleotide.

5. The method of claim 3, wherein the STING agonist is the compound:

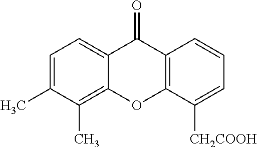

6. The method of any of claims 1 to 5, wherein treating cancer is further defined as reducing the size of a tumor or inhibiting growth of a tumor.

7. The method of any of claims 1 to 6, wherein the STING agonist is administered to the subject at least two, three, four, five, six, seven, eight, nine or ten times.

8. The method of any of claims 1 to 7, wherein said subject is further administered a distinct cancer therapy.

9. The method of claim 8, wherein the STING agonist is administered and then the distinct cancer therapy is administered.

10. The method of claim 9, wherein the distinct cancer therapy is administered within 3 days of the STING agonist.

11. The method of claim 9, wherein the distinct cancer therapy is administered within 24 hours of the STING agonist.

12. The method of claim 9, wherein the distinct cancer therapy is administered within 3 hours of the STING agonist.

13. The method of claim 8, wherein the distinct cancer therapy is administered and then the STING agonist is administered.

14. The method of claim 13, wherein the STING agonist is administered within 3 days of the distinct cancer therapy.

15. The method of claim 13, wherein the STING agonist is administered within 24 hours of the distinct cancer therapy.

16. The method of claim 13, wherein the STING agonist is administered within 3 hours of the distinct cancer therapy.

17. The method of any of claims 8 to 16, wherein said distinct cancer therapy comprises surgery, radiotherapy, chemotherapy, toxin therapy, immunotherapy, cryotherapy or gene therapy.

18. The method of any of claims 1 to 17, wherein the cancer is melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, or hepatocellular carcinoma.

19. The method of claim 18, wherein the cancer is melanoma.

20. The method of any of claims 1 to 19, wherein the cancer is a chemotherapy or radio-resistant cancer.

21. The method of any of claims 1 to 20, wherein the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 150, 200, 250, or 300 μg/kg or mg/kg of the agonist.

22. The method of any of claims 1 to 21, wherein the STING agonist is a non-naturally occurring cyclic dinucleotide.

23. The method of any of claims 1 to 22, wherein the STING agonist is a compound of the formula: 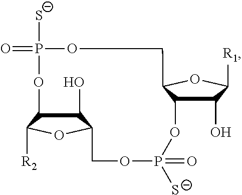
wherein R1 and R2 are each independently any one of 9-purine, 9-adenine, 9-guanine, 9-hypoxanthine, 9-xanthine, 9-uric acid, or 9-isoguanine, or prodrugs or pharmaceutically acceptable salts thereof.

24. The method of claim 23, wherein the compound is in the form of predominantly Rp,Rp or Rp,Sp diastereomers.

25. The method of claim 23, wherein the STING agonist is dithio-(Rₚ, Rₚ)-[cyclic[A(2′,5′)pA(3′,5′)p]] (also known as 2′-5′, 3′-5′ mixed phosphodiester linkage (ML) RR-S2 c-di-AMP or ML RR-S2 CDA)), ML RR-S2-c-di-GMP (ML-CDG), ML RR-S2 cGAMP, or any mixtures thereof.

26. The method of claim 23, wherein the STING agonist is ML RR-S2 CDA.

27. A non-naturally occurring compound of the formula:

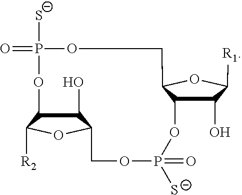

28. The compound of claim 27, wherein the compound is in the form of Rp,Rp diastereomers.

29. The compound of claim 27, wherein the compound is in the form of Rp,Sp diastereomers.

30. The compound of claim 27, wherein the compound is ML RR-S2 CDA, ML RR-S2-CDG, ML RR-S2-cGAMP, or any mixtures thereof.

31. The compound of claim 28, wherein the compound is ML RR-S2 CDA.

32. A method of treating cancer in a subject, comprising administering to the subject an effective amount of a compound in accordance with any of claims 27-31.


SEQUENCE LISTING (13 sequences)

SEQ ID NO: 1 - DNA, 21 bp, Mus musculus
ggaaagaaguguugcgguutt

SEQ ID NO: 2 - DNA, 21 bp, Mus musculus
ggauccgaauguucaaucatt

SEQ ID NO: 3 - DNA, 20 bp, Mus musculus
aacaccggtctaggaagcag

SEQ ID NO: 4 - DNA, 20 bp, Mus musculus
catatttggagcggtgacct

SEQ ID NO: 5 - DNA, 8 bp, Mus musculus
catccagc

SEQ ID NO: 6 - DNA, 20 bp, Mus musculus
caagaggcttgtgatggtca

SEQ ID NO: 7 - DNA, 20 bp, Mus musculus
gcaagtccacggttttcagt

SEQ ID NO: 8 - DNA, 8 bp, Mus musculus
aggagctg

SEQ ID NO: 9 - DNA, 21 bp, Mus musculus
gauuucugcuccuaaugaatt

SEQ ID NO: 10 - DNA, 21 bp, Mus musculus
uucauuaggagcagaaauctt

SEQ ID NO: 11 - DNA, 20 bp, Mus musculus
gaatcttccggagcaaaatg

SEQ ID NO: 12 - DNA, 21 bp, Mus musculus
ggcagttttcacatggtagga

SEQ ID NO: 13 - DNA, 8 bp, Mus musculus
catccagc